(19) United States
(12) Patent Application Publication
(10) Pub. No.: US 2025/0195102 A1
(43) Pub. Date: Jun. 19, 2025

(54) MULTI-PORTAL SURGICAL MARKING GUIDES AND ACCESS INSTRUMENTS

(71) Applicant: Amplify Surgical, Inc., Irvine, CA (US)

(72) Inventors: Andy Wonyong Choi, Irvine, CA (US); Clark Hutton, Carlsbad, CA (US)

(21) Appl. No.: 18/988,467

(22) Filed: Dec. 19, 2024

Related U.S. Application Data
(60) Provisional application No. 63/611,888, filed on Dec. 19, 2023.
(60) Provisional application No. 63/568,701, filed on Mar. 22, 2024.

(51) Int. Cl. A61B 17/3211; A61B 1/313; A61B 17/32; A61B 90/00

(52) U.S. Cl. CPC A61B 17/3211; A61B 90/39; A61B 1/3135; A61B 2017/320044; A61B 2017/320052; A61B 2090/062; A61B 2090/3908; A61B 2090/3966

(57) ABSTRACT

A multi-portal method for treating a subject's spine includes using a marking guide to align spinal incisions. The marking guide can include alignment features, such as radiopaque structures or elements that align with features such as anatomical elements, planes, implants, and/or the like. The marking guide can fit multiple access instruments, such as expandable scalpels, depth guides, surgical instruments, and/or the like. A user can position the access instrument relative to a feature using an alignment feature of the marking guide. The access instruments can be used to create incision sites and prepare entrances for multiple cannulas, cannulas and surgical instruments, multiple surgical instruments, and/or the like. One or more access instruments can be used to enlarge a working space between the entrances for delivery of an interbody fusion implant. The patient's spine can be visualized using endoscopic techniques to view the working space throughout the surgical procedure.

### CROSS-REFERENCE TO RELATED APPLICATIONS

The present application claims the benefit of U.S. Provisional Patent Application No. 63/611,888, filed Dec. 19, 2023, and U.S. Provisional Patent Application No. 63/568,701, filed Mar. 22, 2024, the disclosures of which are incorporated herein by reference in their entireties.

### TECHNICAL FIELD

The present disclosure relates generally to medical systems and, more particularly, to systems, devices, marking guides, and methods for performing multi-portal surgical procedures.

### BACKGROUND

Individuals often suffer from damaged or displaced spinal discs and/or vertebral bodies due to trauma, disease, degenerative defects, or wear over an extended period of time. One result of this displacement or damage to a spinal disc or vertebral body may be chronic back pain. A common procedure for treating damage or disease of the spinal disc or vertebral body may involve partial or complete removal of an intervertebral disc. An intervertebral implant (commonly referred to as an interbody spacer or cage) can be inserted into the cavity created where the intervertebral disc was removed to help maintain height of the spine and/or restore stability to the spine. An interbody spacer may also provide a lordotic correction to the curvature of the spine. An example of an interbody spacer that has been commonly used is a fixed dimension cage, which typically is filled with bone and/or bone growth-inducing materials. Unfortunately, it may be difficult to implant the interbody spacer at the intended implantation site between vertebral bodies. Additionally, conventional surgical techniques can cause a significant amount of trauma at or near the implantation site (e.g., injury to nerve tissue), which can significantly increase recovery time and/or lead to patient discomfort.

Spinal nerve compression can be caused by narrowing of the spinal canal associated with arthritis (e.g., osteoarthritis) of the spine, degeneration of spinal discs, and thickening of ligaments. Arthritis of the spine often leads to the formation of bone spurs, which can narrow the spinal canal and press on the spinal cord. In spinal disc degeneration, inner tissue of the disc can protrude through a weakened fibrous outer covering of the disc and can press on the spinal cord and/or spinal nerve roots. Ligaments located along the spine can thicken over time and press on the spinal cord and/or nerve roots. Unfortunately, spinal nerve compression can cause lower back pain, hip pain, and/or leg pain and may also result in numbness, depending on the location of the compressed nerve tissue. For example, spinal stenosis that causes spinal cord compression in the lower back can cause numbness of the legs. It is difficult to visualize internal tissue when removing tissue, often resulting in injury or removal of nerve tissue. Accordingly, there is a need for improved surgical systems, visualization techniques, and/or related technologies.

### BRIEF DESCRIPTION OF THE DRAWINGS

FIG. 1A is a side view of a multi-portal surgical system and a subject in accordance with an embodiment of the disclosure.

FIGS. 1B and 1C are top views of a marking guide of the multi-portal surgical system of FIG. 1A positioned on a subject.

FIGS. 2A-2D are top views of marking guides in accordance with another embodiments of the disclosure.

FIG. 3A shows a multi-portal surgical system and a subject in accordance with another embodiment of the disclosure.

FIGS. 3B and 3C are front views of an expandable scalpel of the multi-portal surgical system of FIG. 3A.

FIG. 4 is a front view of a scalpel in accordance with an embodiment of the disclosure.

FIGS. 5A and 5B are front views of the scalpel of FIG. 4 with a blade in different positions.

FIGS. 6A-6F are side views of blades in accordance with embodiments of the disclosure.

FIG. 7A shows a multi-portal surgical system and a subject in accordance with an embodiment of the disclosure.

FIG. 7B is an isometric view of a depth guide of the multi-portal surgical system of FIG. 7A.

FIG. 7C shows depth markings on the depth guide of FIG. 7B.

FIG. 8 shows a scalpel and depth guide of a multi-portal surgical system positioned in a subject in accordance with an embodiment of the disclosure.

FIGS. 9A and 9B show a tissue dissector in accordance with an embodiment of the disclosure.

FIG. 9C is an isometric view of a tissue dissector in accordance with an embodiment of the disclosure.

FIG. 9D is a cross-sectional side view of the tissue dissector of FIG. 9C in accordance with an embodiment of the disclosure.

FIG. 10 shows a visualization and working instrument of a multi-portal surgical system in accordance with an embodiment of the disclosure.

FIGS. 11A-11F illustrate surgical steps for performing spinal procedures in accordance with embodiments of the disclosure.

FIG. 12A is a top view of a marking guide with radiopaque alignment features positioned on a subject in accordance with an embodiment of the disclosure.

FIG. 12B is a backside view of the marking guide of FIG. 12A.

FIG. 13A is a top view of the marking guide of FIGS. 12A and 12B.

FIG. 13B is a detailed view radiopaque alignment features of the marking guide of FIG. 13A.

FIG. 14A is a top view of a marking guide with embedded radiopaque alignment features in accordance with an embodiment of the disclosure.

FIG. 14B is a detailed view of an embedded radiopaque alignment feature of FIG. 14A.

FIG. 15 is a top view of a marking guide with radiopaque alignment features in accordance with an embodiment of the disclosure.

FIG. 16 is an isometric view of a muscle detacher in accordance with embodiments of the disclosure.

FIG. 17 is an isometric view of a depth gauge in accordance with embodiments of the disclosure.

FIG. 18A is an isometric view of a spade knife in accordance with embodiments of the disclosure.

FIGS. 18B and 18C are enlarged isometric and side views, respectively, of a blade of the spade knife of FIG. 18A.

FIGS. 19A-19E are front isometric, rear isometric, side, top, and angled top views, respectively, of a root retractor in accordance with embodiments of the disclosure.

FIG. 20 is an isometric view of another root retractor in accordance with embodiments of the disclosure.

FIG. 21 is an isometric view of yet another root retractor in accordance with embodiments of the disclosure.

FIG. 22 is an isometric view of a root retractor bayonet in accordance with embodiments of the disclosure.

FIG. 23 is an isometric view of another root retractor bayonet in accordance with embodiments of the disclosure.

FIG. 24 is an isometric view of yet another root retractor bayonet in accordance with embodiments of the disclosure.

FIG. 25 is an isometric view of a fusion guide in accordance with embodiments of the disclosure.

FIG. 26 is an isometric view of another fusion guide in accordance with embodiments of the disclosure.

FIG. 27 is an isometric view of a scope cannula in accordance with embodiments of the disclosure.

FIG. 28 is an isometric view of another scope cannula in accordance with embodiments of the disclosure.

FIG. 29 is an isometric view of yet another scope cannula in accordance with embodiments of the disclosure.

FIGS. 30A-30C are isometric, side, and front views, respectively, of a cannula in accordance with embodiments of the disclosure.

FIG. 31 is a plan view of a surgical kit in accordance with embodiments of the disclosure.

### DETAILED DESCRIPTION

The following disclosure describes various embodiments of medical systems, devices, kits, and associated methods of use. At least some embodiments are directed to a surgical system or kit providing incision guidance. The system or kit can include multi-portal surgical guides with marking openings and one or more alignment features. The alignment features can include anatomical reference features and/or imaging structures or elements. The imaging structures or elements can align with one or more spinal markers such as anatomical elements of the patient, anatomical planes of the patient, existing implants in the patient, and/or the like. The guides can be incision marking guides with slits used to mark incision sites using a surgical marking pen or skin marker. The slits can also indicate lengths of incisions suitable for receiving instruments, including, surgical and/or access instruments, such as depth guides, dilators, working instruments (e.g., rongeurs, scrapers, etc.), combinations thereof, or the like. The slits can include and/or can be adjacent to the radiopaque structures or elements. For example, an imaging element in the form of a radiopaque lining can surround one or more of the slits. Additionally or alternatively, a radiopaque element can be embedded into or adhered to a surface of the guide adjacent to one or more of the slits.

The instruments can be used to alter tissue (e.g., shape, crush, separate, cut, debulk, break, fracture, or remove tissue), create working spaces, create delivery paths, prepare an implantation site, combinations thereof, or the like. The multi-portal surgical guides can be removed after forming the incisions, working spaces, delivery paths, and/or the like. For example, the surgical guides can be removed from the patient to uses instruments that are larger than the marking openings (e.g., slits). The generally larger instruments, for example, additional access instruments, cannulas, surgical instruments, cameras and can be used to further prepare an implantation site, implant a device, combinations thereof, or the like. The surgical guides can be repositioned on the patient any number times during the procedure to make additional incisions and/or assist with position of instruments (e.g., to realign the instruments along a trajectory, align instruments with anatomy, etc.). The surgical guides can be interchangeable or can be positioned adjacent to one another to extend a length and/or width of a guide and/or the guide's slits. The marking openings and/or slits of the guides are also referred to herein as “incision openings,” “slots,” and/or the like.

Access instruments, surgical instruments, and/or tissue visualization can help a physician identify tissue, remove tissue, remove tissue under visualization, and/or prevent or limit injury or damage to non-targeted organs and tissues. In endoscopic-assisted surgeries, instruments and implantable devices can be precisely positioned using minimally invasive techniques to improve outcomes and reduce recovery times. Such minimally invasive techniques can include, for example, using multi-portal surgical marking guides and access instruments to prepare an incision site and map implantation. Certain details are set forth in the following description and in the figures to provide a thorough understanding of such embodiments of the disclosure. Other details describing well-known structures and systems often associated with, for example, surgical procedures are not set forth in the following description to avoid unnecessarily obscuring the description of various embodiments of the disclosure.

### A. Overview

At least some embodiments are directed to multi-portal surgical systems configured to treat patients with, for example, nerve compression, damaged or displaced spinal features (e.g., spinal discs and/or vertebral bodies), or other conditions. For example, the surgical systems can be used to reduce or eliminate nerve compression, implant a fixed or expandable interbody device (e.g., devices to space apart vertebral bodies, restore stability of the spine, provide lordotic correction, etc.), perform discectomies, perform microdiscectomies, perform laminotomies, combinations thereof, or other surgical procedures. One or more multi-portal surgical guides can be used to determine the number and/or location of insertion sites based on physical characteristics of the subject. A physician can select marking openings of the surgical guide(s) to achieve target instrument trajectories relative to a working zone or target site.

In some embodiments, a multi-portal surgical method can include positioning a marking guide along a subject in a selected direction relative to the subject. For example, a plurality of incision openings of the marking guide can be spaced apart in, for example, a superior/inferior direction relative to the subject. Additionally or alternatively, the plurality of incision openings of the marking guide can be spaced apart, in a medial/lateral direction or another direction relative to the subject. A user can select how to use the marking guide based on one or more physical characteristics of the subject. The user can select incision openings corresponding to a quantified physical characteristic (e.g., body mass index, weight, etc.) of the subject. The marking guide can have any number of incision openings corresponding to different body types. This allows the marking guide to be used to determine incision sites that allow similar surgical techniques to be performed on different patients. For example, the marking openings can be labeled according to BMI, depth of subcutaneous fat, and/or other physiological parameters. In some procedures, for example, a user can select a first incision opening based on the subject's body mass index. A user can image (e.g., via X-ray, fluoroscopy) the anatomy and marking guide to confirm the position of the first incision relative to an alignment feature. The alignment features can include anatomical reference features and/or radiopaque structures or elements that orient the marking guide relative to anatomical elements, planes, implants, and/or the like. The user can then create a first incision along the subject using the selected first incision opening by, for example, marking the first incision site using a surgical marking pen, skin marker, and/or a scalpel to cut tissue at the first incision site. The user can create a second incision along the patient using a second incision opening. The user can then deliver instruments through the first and second incisions. For example, a working instrument can be delivered through the first incision towards a working zone in the subject. A visualization instrument can be delivered through the second incision such that the visualization instrument provides viewing of a distal end of the working instrument while both the working instrument and the visualization instrument are angled toward the working zone.

In some embodiments, multi-portal surgical marking guides can be configured to align and create incisions. The multi-portal surgical marking guides can be configured to fit multiple instruments, including, access instruments, such as scalpels, depth guides, surgical instruments, and/or the like. The access instruments can be used to create incisions and prepare entrances for multiple cannulas, cannulas and surgical instruments, multiple surgical instruments, and/or the like. For example, fascial tissue can be cut, split, and/or removed using one or more access instruments inserted through the marking guide. The multi-portal surgical marking guides can be coupled (e.g., coupled via an adhesive, pressure-sensitive adhesive, etc.) to the patient's body to, for example, reduce, inhibit, or limit movement of the guide as incision sites are marked (e.g., with a surgical marking pen or skin marker), incisions are made, etc. The marking guide can be removed, and one or more additional access instruments are used to clear a working area for surgical instruments. Cannulas (e.g., split cannulas, non-split tubular cannulas, etc.) can also be used to position instruments and/or access tissue.

A multi-portal surgical marking guide and the access instruments can be used to create delivery paths sized for cannulas and surgical instruments. Visualization instruments in the patient can also be inserted through the entrances created using the multi-portal surgical marking guide and the access instruments. The visualization instruments can provide viewing of working spaces, tissue contributing to the nerve compression, and tissue removal instruments. In some embodiments, the multi-portal surgical marking guide can be repositioned on the patient or replaced with additional surgical marking guide(s) based on the viewing capability the visualization instrument, surgical steps to be performed, etc. For example, the viewing can reveal obstacles or elements in the working space. The user can reposition or replace the surgical marking guide to make one or more additional incisions on the patient that repositions the instruments in the working space. The viewing can reveal that at least one of the instruments is not positioned along the instrument's target trajectory relative to a working zone or target site. The user can reposition or replace the surgical marking guide to make an additional incision on the patient that repositions the instrument along the target trajectory.

Access instruments can be selected based on the location and/or size of a desired working space. In some procedures, an initial incision can be made with a scalpel. The scalpel can include one or more deployable blades configured to pass through the marking guide. The one or more deployable blades can, for example, articulate laterally and/or expand outwardly to create wider and/or longer cuts in the subcutaneous tissue to enlarge the working zone with or without enlarging the skin incisions. In some procedures, a depth guide is used to measure the depth of the cuts, size of the working space, etc. In some embodiments, a scalpel can include a lever and a deployable tissue-dissector movable to create a larger working space in the patient. For example, the lever can deploy the tissue-dissector (e.g., a blade, a tip, a wedge, and/or the like) that moves laterally outwards to split additional tissue and create the larger working zone.

Surgical instruments can be used with or without the marking guide to sequentially access and remove additional tissue. The number and type of access instruments used to create or access of the working area can be selected based on the location (e.g., depth) of the tissue, anatomical structures surrounding access paths and/or targeted tissue, and/or configuration of instrument(s) to be used in the procedure. Some access instruments can be used to create a relatively large subcutaneous working spaces relative to the initial incision made in the skin (i.e., the incision can be significantly smaller than the size of the working space within the patient).

In some embodiments, multi-portal surgical techniques can be used to alter tissue at different locations along the spine. The multi-portal surgical techniques can include the use of multi-portal surgical marking guides, multi-portal endoscopic techniques, and/or the like. Bony features (e.g., facets and surrounding bone) of vertebrae can be removed to perform, for example, transforaminal procedures. Fascial tissue can be removed and/or altered to visualize an implantation site. The implantation site can be prepared by performing a discectomy, an interbody preparation procedure, and/or the like. Surgical marking guides can be selected based on the procedure to be performed. For example, a surgical marking guide for multi-level lumbar fusion procedures can have sets of marking openings for each targeted spinal level. By way of another example, multi-portal surgical marking guides can be specifically configured to perform multi-portal spinal decompression procedures. A physician can select one or more of the surgical marking guides from a set of surgical marking guides in a kit based on the procedure. Additionally, the surgical marking guides can indicate a target range of instrument positions so that the physician can select the marking guide based on the surgical techniques to be used.

Multi-portal endoscopy-assisted methods can include performing at least a portion of a surgical procedure using a first portal site. The first portal site can serve as a working portal for working instruments. At least a portion of the surgical procedure uses an endoscope positioned via a second portal site (e.g., a visualization portal) spaced apart from the first portal site. The spacing can be selected based on location and accessibility of the treatment site(s), whether along the spine or at another location. For example, a multi-portal surgical marking guide can be used to position and/or orient the first portal site along a spine (e.g., parallel to sagittal plane). Additionally or alternatively, one or more access instruments can be used to create incisions that align with the multi-portal surgical marking guide. Instruments can also be used to alter tissue (e.g., shape, crush, separate, cut, debulk, break, fracture, or remove tissue), create working spaces, create delivery paths, prepare an implantation site, and/or the like. The second portal site can be spaced apart from the first portal site to allow equipment (e.g., cannulas, endoscopes, working instruments, etc.) to be directed generally toward a working space within the subject. The multi-portal surgical marking guide in combination with one or more access instruments can be used to orient and create the second portal site or any additional sites. The first portal site and the second portal site are also referred to as a “first entrance” and a “second entrance”, respectively, herein.

In some decompression procedures, surgical steps can minimize or reduce pressure applied to nerve tissue and can include removing tissue contributing to stenosis, tissue pushing against nerve tissue, bulging sections of intervertebral cartilage disc, and/or the like. For example, tissue can be removed to enlarge an epidural space to reduce spinal cord compression. A multi-portal surgical marking guide can be configured to assist with positioning decompression instruments to perform such steps.

In some aspects, techniques described herein relate to a multi-portal method including, for example, placing a multi-portal surgical marking guide on a patient and aligning it with one or more spinal markers, anatomical features, anatomical planes, and/or preexisting implants (e.g., pedicle screws, cages, interspinous spacers, etc.). The marking guide can include anatomical reference features and/or radiopaque structures or elements to assist with positioning. One or more access instruments can be used to create a first entrance through the subject's skin. The one or more access instruments can then be used to create a second entrance through the subject's skin. The second entrance can be spaced apart from the first entrance using the multi-portal surgical marking guide. The multi-portal surgical marking guide can align the first entrance and the second entrance to be generally parallel or perpendicular to a sagittal plane of the patient, in a superior-inferior direction, in a medial-lateral direction, or other direction. Additionally, or alternatively, the first entrance and the second entrance can be used to orient cannulas and/or instruments used throughout a surgical procedure.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

### B. Multi-Portal Surgical Systems

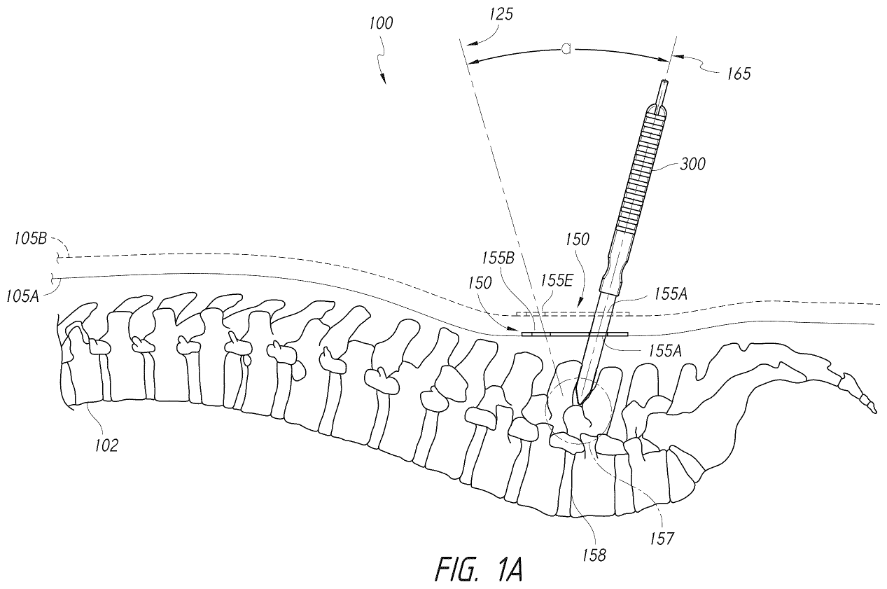

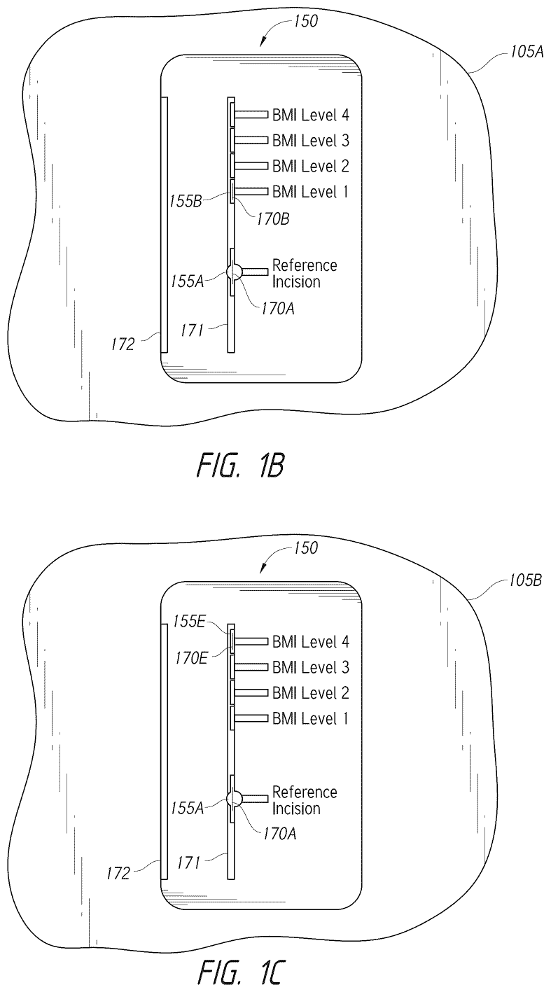

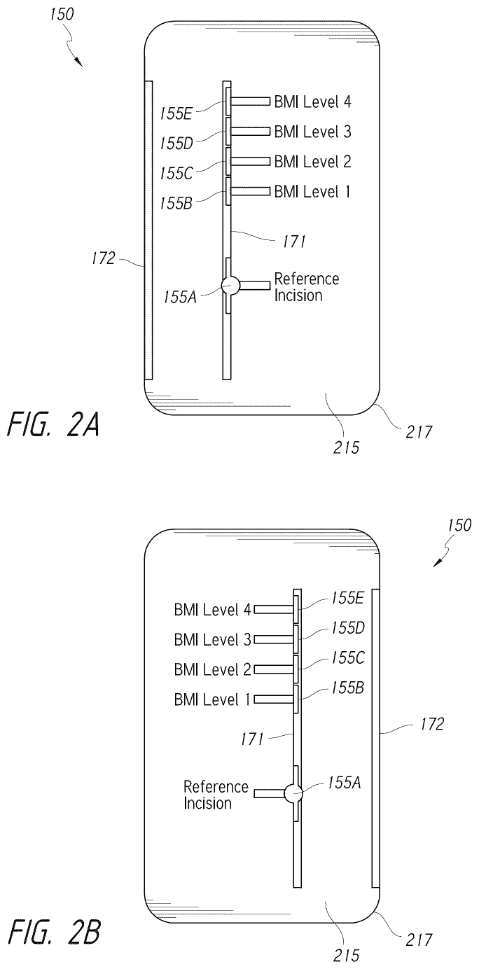

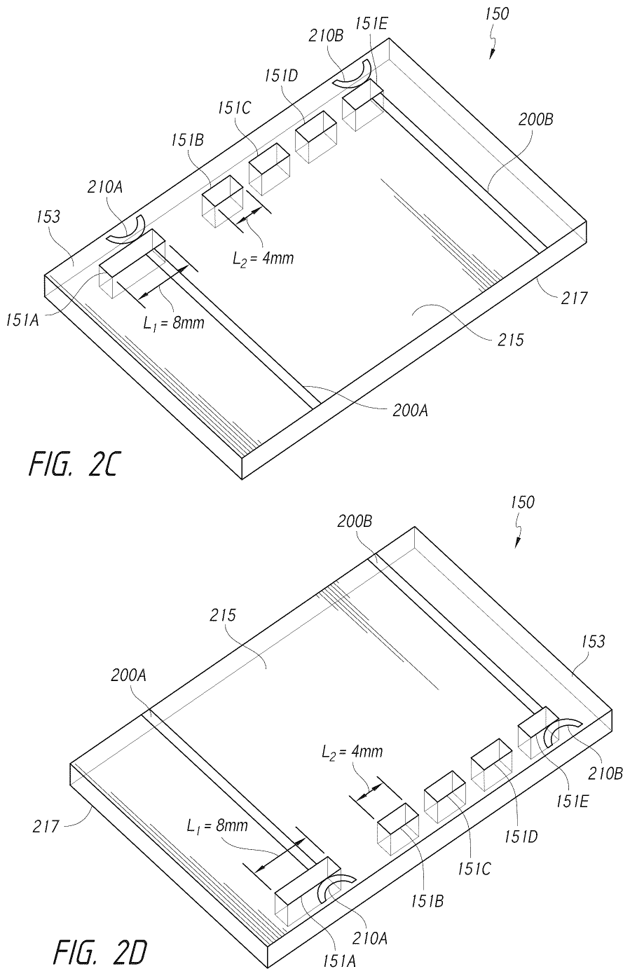

FIG. 1A is a side view of a components of a multi-portal surgical system 100 (“system 100”) positioned along a human subject's spine 102 in accordance with an embodiment of the disclosure. The system 100 can include a multi-portal surgical marking guide 150 (“marking guide 150”). The marking guide 150 can be positioned along the human subject's skin 105A and can be configured to identify portal sites, to align incisions, and/or to hold instruments (e.g., scalpels, depth guides, surgical instruments, combinations thereof, etc.). For example, as shown, the marking guide 150 can be used to align and/or orient an expandable scalpel 300 for initial incision. The marking guide 150 can be used to plan surgical procedures for different types of patients. For example, the marking guide 150 is shown on the skin 105A of a patient having a relatively low body mass index such that the marking guide 150 is relatively close to the spine 102. The illustrated skin 105B (shown in dashed line) illustrates the skin position of a patient having a relatively high body mass index such that the skin 105B is positioned further away from the spine 102 due to thicker tissue layers underlying the skin 105B. For example, the skin 105A can be of a human subject with a low body mass index (e.g., BMI 15-25, BMI less than 20, BMI less than 25, etc.). The skin 105B can be of a human subject with a higher BMI (e.g., BMI greater than 25, 30, 35, 40, 45, 50, etc.). The same marking guide 150 can be used to define portal sites on these different types of patients to achieve targeted instrument trajectories, such as trajectories 125, 165. Example features of the marking guide 150 are discussed in connection with FIGS. 1B-2D.

FIGS. 1B and 1C are top views of the marking guide 150 positioned on the skin 105A of the subject. The scalpel 300 (not shown in FIGS. 1B and 1C) and/or other instruments can be aligned with an elongated opening or slot 171 on the marking guide 150. The marking guide 150 can have multiple marking labels corresponding to multiple incision sites or openings (e.g., incision markings or openings 155A-155E as discussed in connection with FIGS. 1B-2B). Each of the incision openings 155A, 155B, 155E shown in FIGS. 1A-1C can have a corresponding marking label (e.g., illustrated as the reference incision and BMI levels in FIGS. 1B and 1C). The number of incision openings and marking labels can be chosen depending on the surgical procedure to be performed and/or the treatment site(s). The depth from the skin 105A, 105B to the spine 102 can vary between subject based on multiple factors (e.g., BMI, depth of subcutaneous fat, etc.). The incision openings 155A-155E and corresponding marking labels can be used to align the marking guide 150 specific to the subject based on the subject's BMI (and/or another quantifiable characteristic) while maintaining constant trajectories towards the vertebral space. A human subject's BMI can be calculated by dividing the subject's body mass by the square of their height. BMI can be expressed in units of kg/m² and can serve as an indicator of the subject's body fat content. Techniques for determining body fat content and/or calculating BMI can also include a skin fold test, underwater weighing, dual-energy X-ray absorptiometry (DXA), bioelectrical impedance measurements, and/or the like. It is appreciated that in other embodiments, the incision markings or openings 155A-155E and the corresponding marking labels can be associated with different degrees of a different physiological parameter.

Referring now back to FIG. 1A, trajectories 125 and 165 depict alignment/orientations of instruments to access a working zone or space 157 (“working zone 157”), disc 158, intervertebral space, vertebral endplates, etc. For the skin 105A, the incision opening 155B can be used to position instruments along the trajectory 125. For the skin 105B, the incision opening 155E can be used to position instruments along the same trajectory 125. The distance between the incision openings 155E, 155A is greater than the distance between the incision openings 155B, 155A to compensate for the subject's skin 105B surface being further away from the subject's spine 102 than the skin 105A. In some embodiments, the trajectories 125, 165 can be at an angular orientation selected by a user (e.g., a physician, surgeon, member of surgical team, etc.) based on the desired positioning of instruments. For example, the trajectories 125, 165 can define an angle a equal to or less than, for example, 10 degrees, 25 degrees, 35 degrees, 45 degrees, 50 degrees, 60 degrees, or ranges of such angles (e.g., within a range of 25 degree to 60 degrees). The angle a can be selected for enhanced visualization of instruments and/or tissue in the working portal throughout the procedure.

Referring to FIGS. 1B and 1C, the marking guide 150 can have features to help keep the marking guide 150 generally parallel to the subject's sagittal plane or midline. For example, a plane alignment feature 172 can extend in a lengthwise direction on the marking guide 150 to orient the marking guide 150, and thus the incisions 170, to be generally parallel to midsagittal plane and/or supraspinous ligament of the patient. In some embodiments, a physician can visually align the plane alignment feature 172 to generally overlay the patient's supraspinous ligament. The number, configuration, position, and orientation of alignment features can be selected based on the desired alignment of the marking guide. Thus, a longitudinal axis of the elongated slot 171 can be kept generally parallel to the midsagittal plane of the subject during use. This alignment appropriately orients the elongate incision sites made with the marking guide 150 and/or placement of instruments within the marking guide 150. In some embodiments, the marking guide 150 can be coupled (e.g., coupled via an adhesive) to the skin 105A to, for example, reduce, inhibit, or limit movement of the marking guide 150 when marking skin, cutting tissue, etc. Additionally, or alternatively, the marking guide 150 can be used to hold one or more access instruments as the access instruments are used to create a working space.

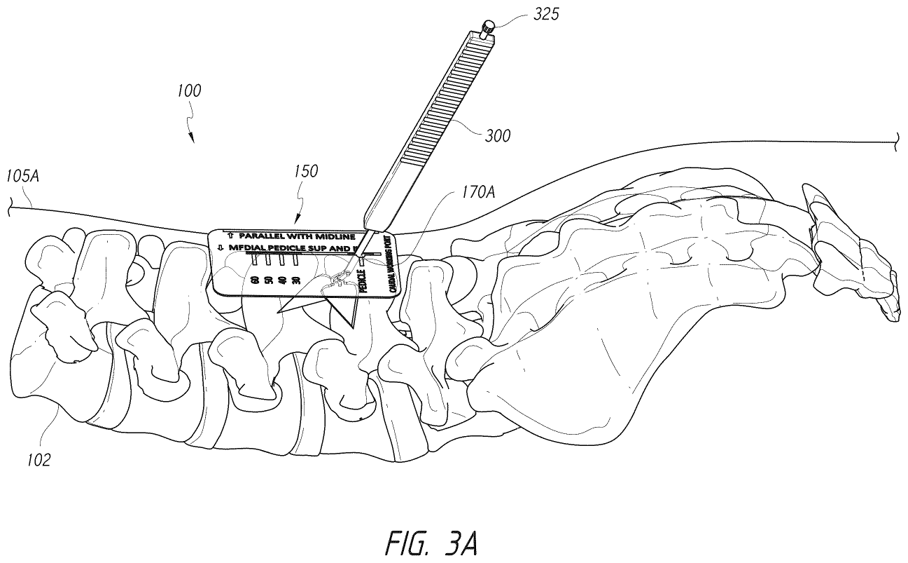

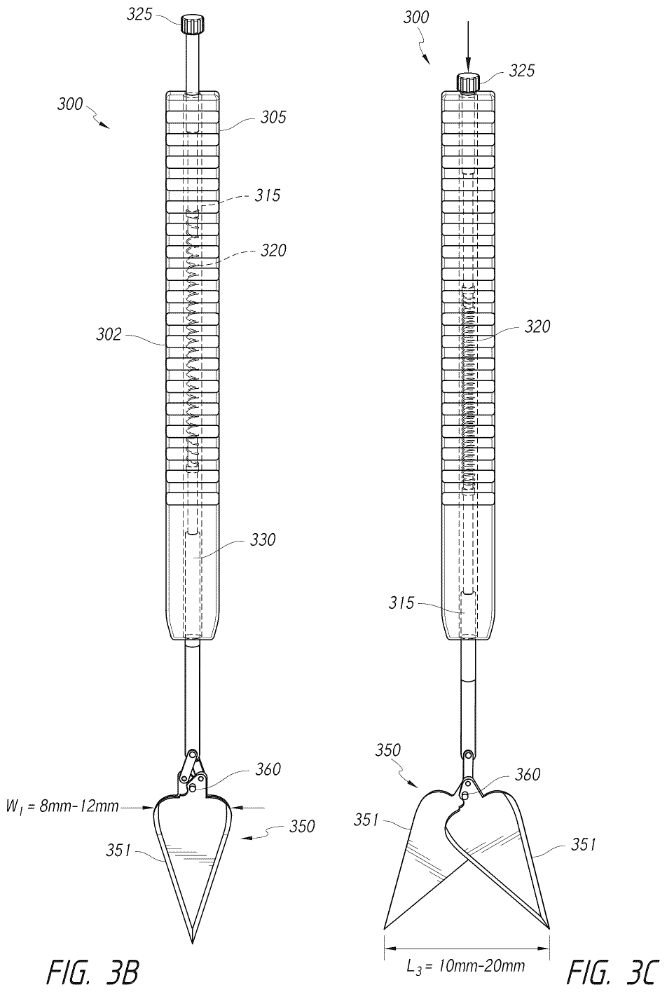

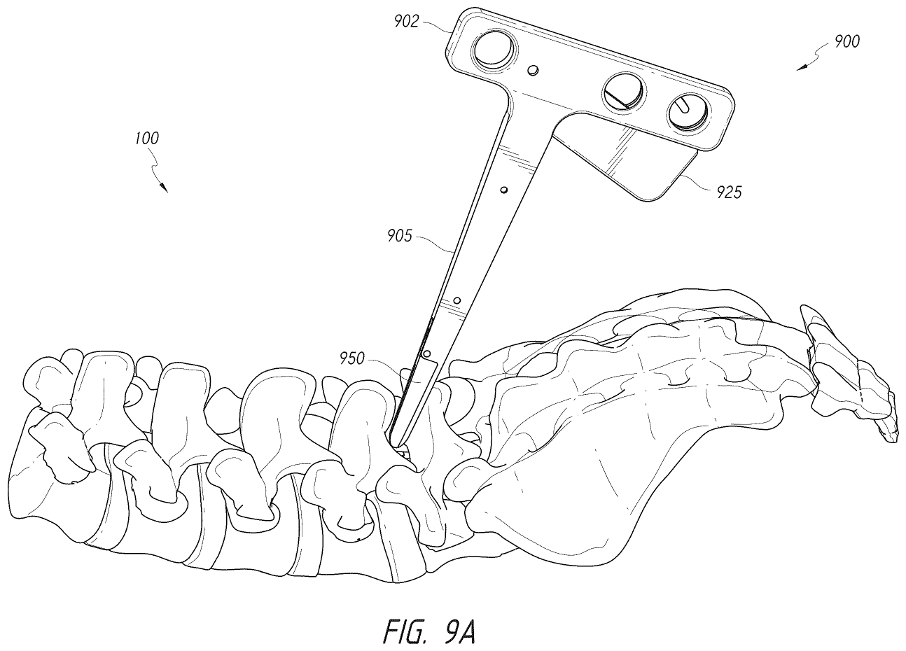

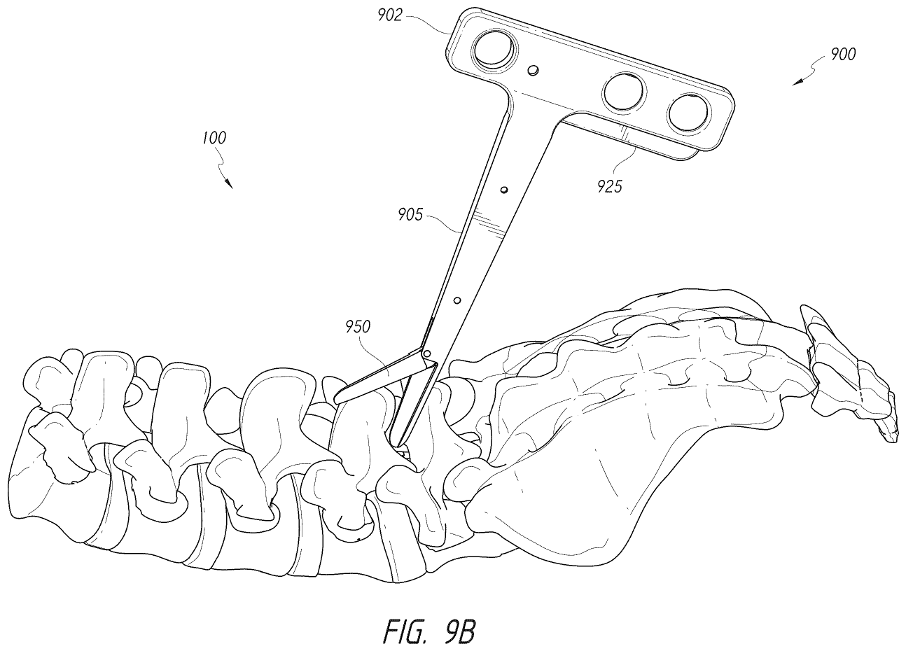

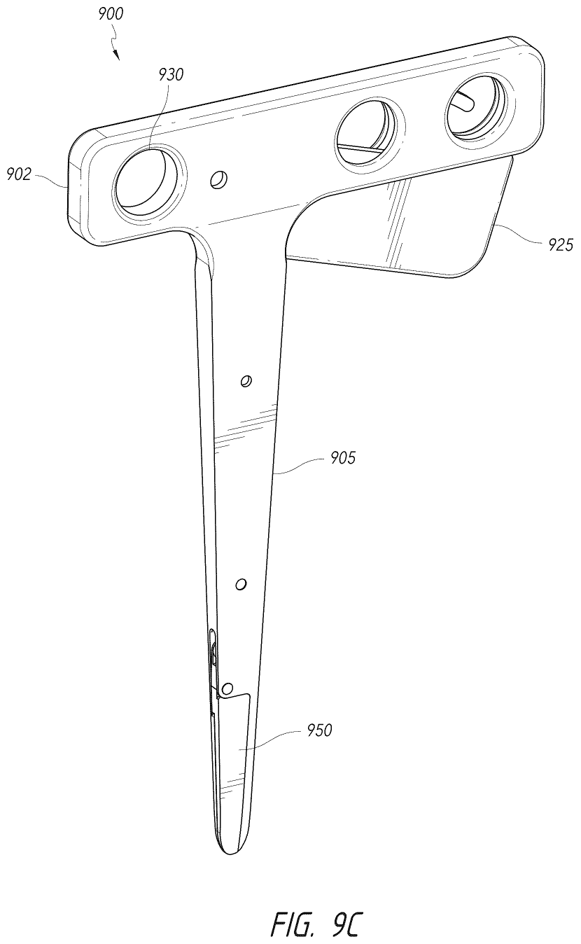

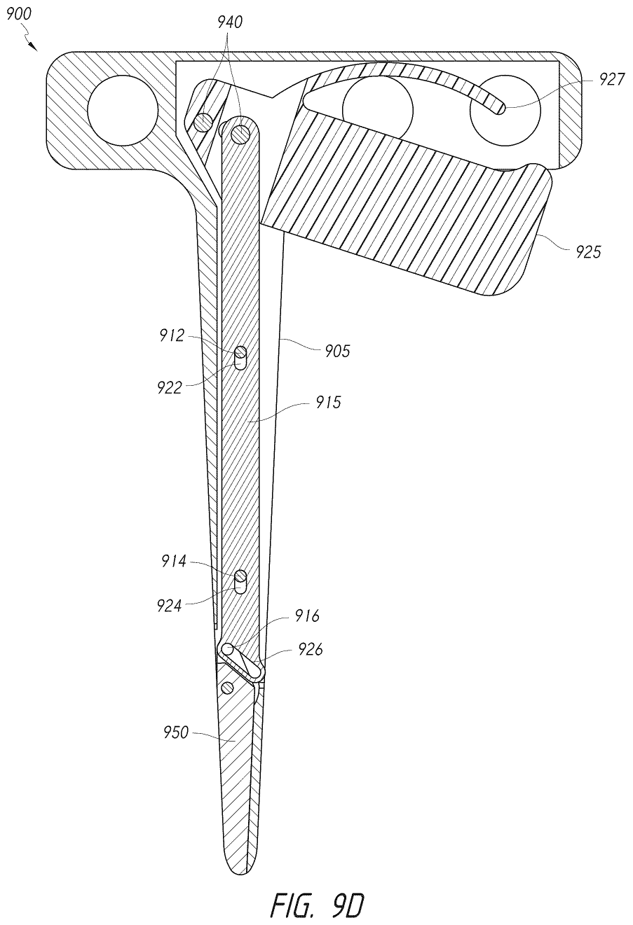

The access instruments (e.g., the scalpel 300 shown in FIG. 1A) can be used to create incisions, alter tissue (shape, crush, separate, cut, debulk, break, fracture, or remove tissue), create working spaces, create delivery paths, prepare an implantation site, combinations thereof, or the like. The access instruments can also be used once the marking guide 150 is removed. For example, the marking guide 150 can be used to align, mark, and/or make initial incisions (as shown in FIGS. 1A-1C) and clear fascial tissue near the incision site (as shown in FIGS. 3A-3C). The marking guide 150 can be removed, and one or more additional access instruments can be used to clear additional fascial tissue (as shown in FIGS. 9A-9D). In some embodiments, this creates a generally larger entrance for cannulas, surgical instruments, visualization instruments, and/or the like. The access instruments used to prepare a working space are discussed in more detail with reference to FIGS. 3A-19.

The marking guide 150 can be made, in whole or in part, of one or more polymers (e.g., transparent polymers for viewing underlying skin, opaque polymers, etc.), composites, plastics, metals, layers (e.g., protective layers, adhesive layers, etc.) or materials suitable for contacting instruments and the skin. In some embodiments, the marking guide 150 can be made of materials or include radiopaque markers compatible with one or more visualization techniques (e.g., fluoroscopy, MRI imaging, CT imaging, direct visualization, etc.). For example, the elongated slot 171 and/or the incision openings 155A-155E can be lined with a radiopaque material for visualization via fluoroscopy.

Referring still to FIGS. 1B and 1C, incisions 170A, 170B, and 170E (“incisions 170”) made using a scalpel (e.g., the expandable scalpel 300 of FIG. 1A in the elongated slot 171) at incision opening 155A (e.g., reference incision), 155B (e.g., BMI Level 1), and 155E (e.g., BMI Level 4), respectively, are shown. The lengths of the incisions 170 can be selected based on the size of the instruments. Example incisions 170 can have lengths generally between 2 mm-6 mm, 4 mm-8 mm, etc. This illustrates how the marking guide 150 can be used on a variety of patients (e.g., patients of different sizes, builds, etc.) while maintaining instruments' positioning relative to the working space 157.

FIG. 2A and 2B are top views of a marking guide 150 in accordance with an embodiment of the disclosure. Referring to FIG. 2A, the marking guide 150 is configured with an elongated slot 171 on the left side of a top face 215. Incision markings or openings 155A-155E can be to the left of the elongated slot 171 of the marking guide 150 with marking labels (e.g., the reference incision, BMI levels, etc.) to the right of the elongated slot 171. FIG. 2B is a mirrored version of the marking guide 150 of FIG. 2A, with the elongated slot 171 on the right side of the top face 215. As shown in FIG. 2B, the incision markings or openings 155A-155E can be to the right of the elongated slot 171 on the marking guide 150 with marking labels (e.g., the reference incision, BMI levels, etc.) to the left of the elongated slot 171. In operation, the marking guide 150 of FIGS. 2A and 2B can be used to make incisions for accessing the left side and the right side of a lumbar spine, respectively.

Referring to FIGS. 2A and 2B collectively, the elongated slot 171 can extend from the top face 215 to a bottom skin-contacting face (not illustrated). The position of the drawn incision markings 155A-155E within the elongated slot 171 can generally correspond to the same positions as slots 151A-151E described in more detail below with reference to FIGS. 2C and 2D. In addition, the slots 151A-151E (as shown in FIGS. 2C and 2D) can be different from the incision openings on the marking guide 150. The marking guide 150 can also include guides and/or anatomical reference elements that are generally similar in configuration and in operation to the guides 200A, 200B and the anatomical reference elements 210A, 210B (as shown in FIGS. 2C and 2D).

FIGS. 2C and 2D are isometric views of a marking guide 150 in accordance with an embodiment of the disclosure. The marking guide 150 has a flexible planar body 153 configured with multiple openings in the slots 151A-151E. FIG. 2D is a mirrored version of the marking guide 150 of FIG. 2C, with the slots 151A-151E positioned along the right side of the top face 215. In operation, the marking guide 150 of FIG. 2C can be used to make incisions on the left side of a patient's spine whereas the marking guide 150 of FIG. 2D can be used to make incisions on the right side of the patient's spine. The slots 151A-151E can extend from the top face 215 to a bottom face 217 to allow one or more instruments (e.g., scalpels, depth guides, surgical instruments, etc.), ink pens, and/or the like to reach the subject's skin (skin omitted for clarity).

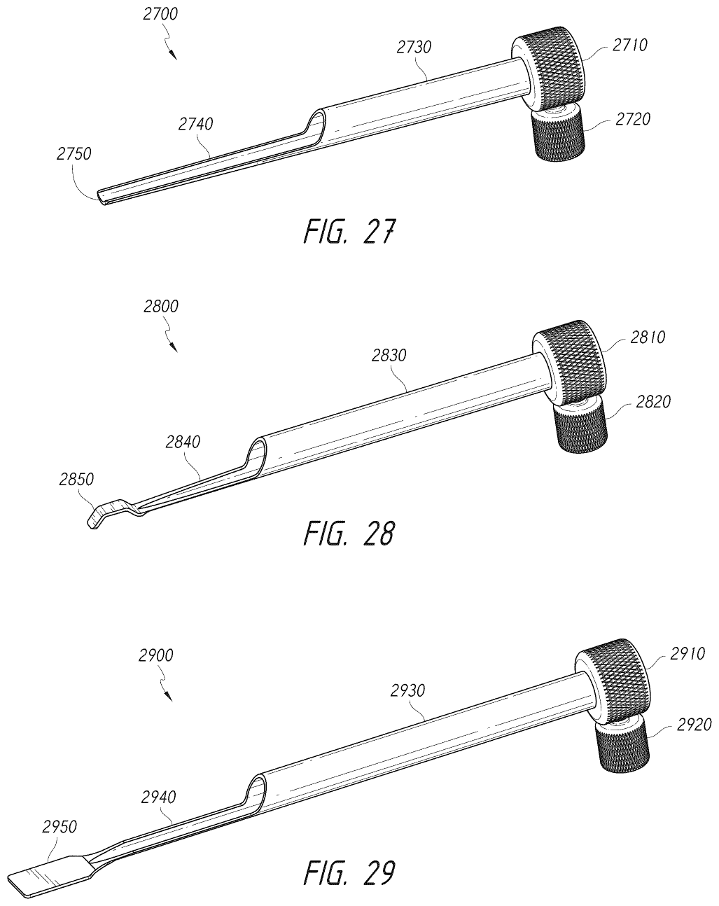

In some embodiments, the slot 151A is longer in length than the slots 151B-151E. For example, the slot 151A can be 8 mm in length (L1) while the slots 151B-151E can be 4 mm in length (L2). In some embodiments, the slot 151A serves as an incision site for a working portal in which larger working instruments are inserted. The working instruments can include, without limitation, dilators, tissue dissectors, scalpels, knives, tissue detachers, retractors, ablation devices, bayonets, etc. Example working instruments are discussed in connection with, for example, FIGS. 1A, 3B-9D, and 16-26. At least some of the surgical procedures described herein use an endoscope positioned in a visualization portal. The slots 151B-151E can serve as an incision site for the visualization portal, such that an endoscope or working instrument can be inserted spaced apart from the slot 151A. The endoscope can be inserted directly through the incision, through a cannula positioned in the incision (e.g., split cannulas, tubular cannulas, cannula 2700 discussed in connection with FIGS. 27-29, etc.), or the like. The spacing can be selected based on location and accessibility of the treatment site(s), whether along the spine or at another location. For example, incision sites or portals can be spaced apart to allow equipment (e.g., cannulas, endoscopes, working instruments, etc.) to be directed generally toward a working space within the subject. The working portal and visualization portal can be described in more detail with reference to FIG. 10.

The marking guide 150 can include guides 200A, 200B (“guides 200”) and anatomical reference elements 210A, 210B (“anatomical reference elements 210”). The guides 200 can be generally parallel to the transverse plane and/or the vertebral space. A physician can mark the skin subjacent to the end of the guides 200 and/or use the guides 200 for alignment with anatomical features, such as a top or bottom of the spinous process. In some procedures, the guides 200 can be oriented in the transverse direction to facilitate superior/inferior alignment of the slots 151A-151E. Similar to the slots 151B-151E, the guides 200 can be slots and configured to allow, for example, an ink pen to reach the subject's skin (skin omitted for clarity). Additionally, or alternatively, the anatomical reference elements 210 can be used to align the marking guide 150 when placed on the subject's skin. The anatomical reference elements 210 can also be parallel to the transverse plane and/or the vertebral space and can be configured to mate and/or align with one or more anatomical reference elements near the treatment site(s). For example, the guide 200A and/or the anatomical reference element 210A can align with the edge of the slot 151A and the spinous process, facet joint, etc. The guide 200B and/or the anatomical reference element 210B can align between, for example, the two vertebral bodies near the treatment site. The guides 200 and/or the anatomical reference elements 210 can also be used to vertically align the marking guide 150.

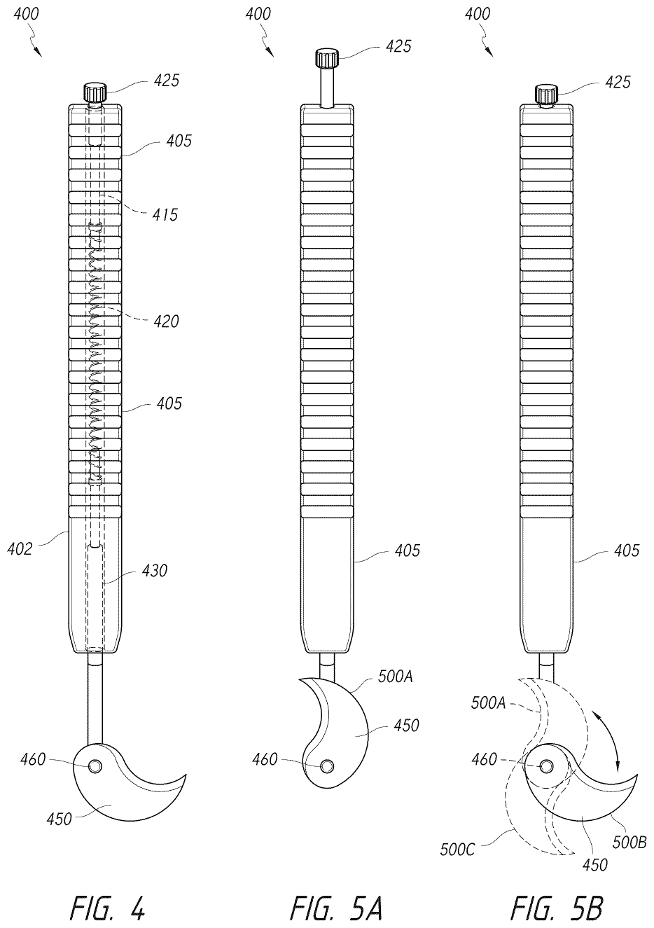

FIG. 3A is a side view of a multi-portal surgical system in accordance with an embodiment of the disclosure. The system 100 of FIG. 3A is generally similar to the system 100 of FIG. 1A except the expandable scalpel 300 is now in a deployed configuration. The scalpel 300 in an undeployed configuration can be inserted through the marking guide 150 at the incision 170A (e.g., between 4 mm-8 mm in length). The scalpel 300 can be advanced past the fascia. A trigger 325 of the scalpel 300 can be used to deploy an expandable blade assembly (e.g., expandable blade assembly 350 of FIGS. 3B and 3C) of the scalpel 300, thereby expanding the scalpel 300 laterally outward as shown. In some embodiments, the blade assembly can expand to create a generally larger cut in the subcutaneous tissue, thereby expanding the working zone, such as a superior-inferior extending opening or working space, as described in more detail with reference to FIGS. 4A-6F.

FIGS. 3B and 3C are front views of the expandable scalpel 300 including a handle or body 305, the trigger 325, and an expandable blade assembly 350. The body 305 can be configured to be manually held by a user (e.g., surgeon, physician, member of a surgical team, etc.) during the surgical procedure. The body 305 can include a connector or driver 315 configured to compress a biasing element 320 when the trigger 325 is activated (e.g., by mechanically pushing the trigger 325 from an extended position of FIG. 3B to a depressed position of FIG. 3C). The biasing element 320 can include one or more springs, such as torsional springs, helical springs, coil springs, etc. The expandable blade assembly 350 can include blades 351 coupled together by a coupler assembly 360 (e.g., hinges, pivots, joints, links, etc.) at their proximal ends. In some embodiments, the blades 351 have a maximum width between W1=8 mm-12 mm. In some embodiments, when the trigger 325 is activated, a member 330 pushes down on the expandable blade assembly 350, causing the blades 351 to rotate outwards. The blades 351 in this position can be used to cut a larger length of tissue and create a generally larger working space in the patient, as discussed herein.

FIGS. 3B and 3C show expandable blade assembly 350 in an undeployed configuration and deployed configuration, respectively. In the undeployed configuration, the blades 351 are generally parallel to the body 305 with the tip of the blades 351 facing distally. In the deployed position, the tip of the blades 351 splay and/or spread out in different directions to create a working space with a length (L3) between 10 mm-20 mm.

When the scalpel 300 is in the deployed position, a user can move (e.g., pull) the trigger 325 to revert the biasing element 320 to an uncompressed position (e.g., via tension, etc.). This, in turn, reverts the expandable blade assembly 350 back to the undeployed position. The process can be repeated any number of times until a suitable working space has been formed for the surgical procedure being performed. The working space can be deemed cleared or formed by one or more user's (e.g., a surgeon, a member of a surgical team, combination thereof, or the like).

FIG. 4 is a front view of an expandable scalpel 400 in accordance with an embodiment of the disclosure. The scalpel 400 as described in FIGS. 4 and 5A-5B is generally similar in configuration and in operation to the scalpel 300 of FIGS. 3A-3C except the scalpel 400 includes a singular curved or crescent-shaped deployable blade 450. The scalpel 400 includes a handle or elongate body 405, a trigger 425, and the blade 450. The body 405 can include a connector or driver 415 configured to compress a biasing element 420 when the trigger 425 is activated (e.g., as described in FIGS. 3A-3C). In some embodiments, when the trigger 425 is activated, a member 430 pushes down on the blade 450, causing the blade 450 to rotate outwards. The body 405 exterior can be made, in whole or in part, of one or more polymers, composites, plastics, metals, or materials suitable for contacting a user's skin. Moreover, one or more of the components of the scalpel 400 can be disposable and can be made from metal, polymer, ceramic, composite, or other biocompatible and sterilizable material.

FIGS. 5A and 5B are front views of the expandable scalpel 400 in FIG. 4. The blade 450 of the expandable scalpel 400 can create a vertically enlarged working space in a patient. In the undeployed position 500A of FIG. 5A, the blade 450 is generally parallel to the body 405 (as described above with reference to FIG. 4) with the tip of the blade 450 facing proximally. Now referring to FIG. 5B, the blade 450 of the expandable scalpel 400 is in a laterally deployed position 500B such that a tip of the blade 450 faces an axis generally perpendicular to the body 405. In operation, the expandable scalpel 400 can be placed into a working portal in the undeployed position 500A. When deployed, the blade 450 is rotated (e.g., rotated laterally outwards) at an angle (e.g., 45 degrees, 90 degrees, 180 degrees, 270 degrees, etc.) into tissue. As shown in FIG. 5B, the blade 450 can also be in a second undeployed position 500C. In the second undeployed position 500C, the blade 450 is generally parallel to the body 405 with the tip of the blade 450 facing distally. In some embodiments, the second undeployed position 500C can be used to make skin incisions (e.g., between 4 mm-8 mm in length) on the skin (as described in FIG. 1A). Once inside the patient, the trigger 425 can be used to deploy the blade 450 to the laterally deployed position 400B. In some embodiments, the scalpel 400 can be used to alter tissue (e.g., scrape soft tissue off bone, split muscles, remove tissue, etc.) and/or create working space for instruments (surgical instrument, cannulas, combinations thereof, or the like).

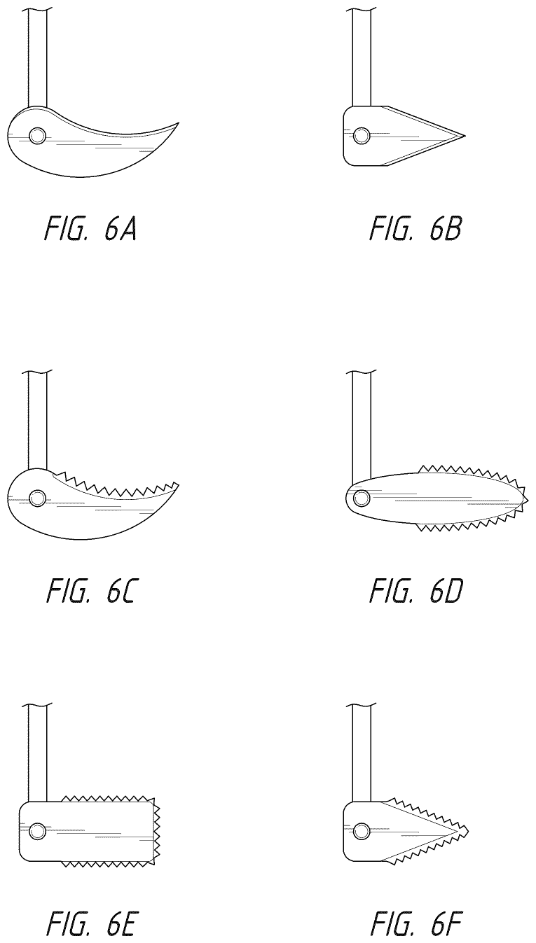

FIGS. 6A-6F are side views of blades for scalpels in accordance with embodiments of the disclosure. The deployable blades can be hook shapes, ovular shapes, rectangular shapes, triangular shapes, combinations thereof, or the like. Referring now to FIGS. 6C-6F, deployable blades can also be configured with teeth, serrated portions, and/or the like. The configuration of the blades can be selected based on the surgical steps to be performed.

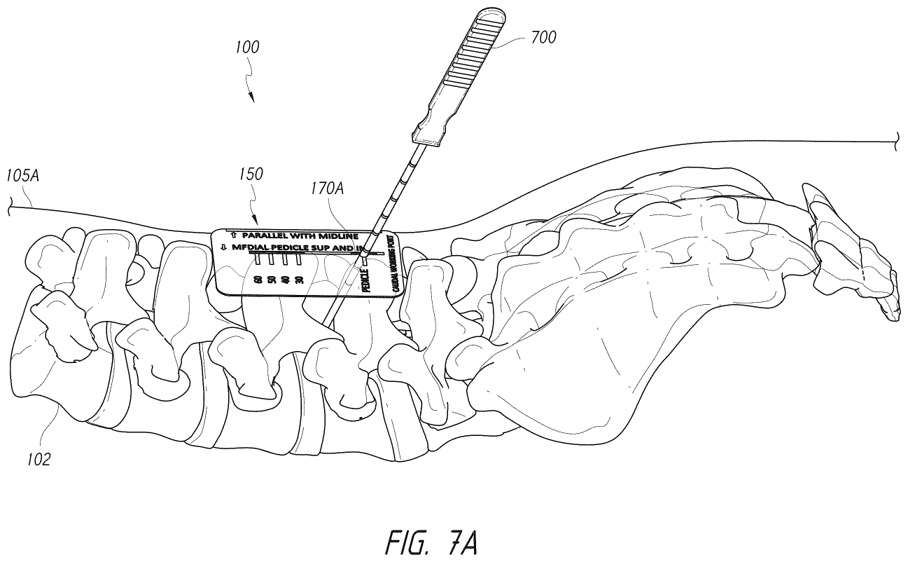

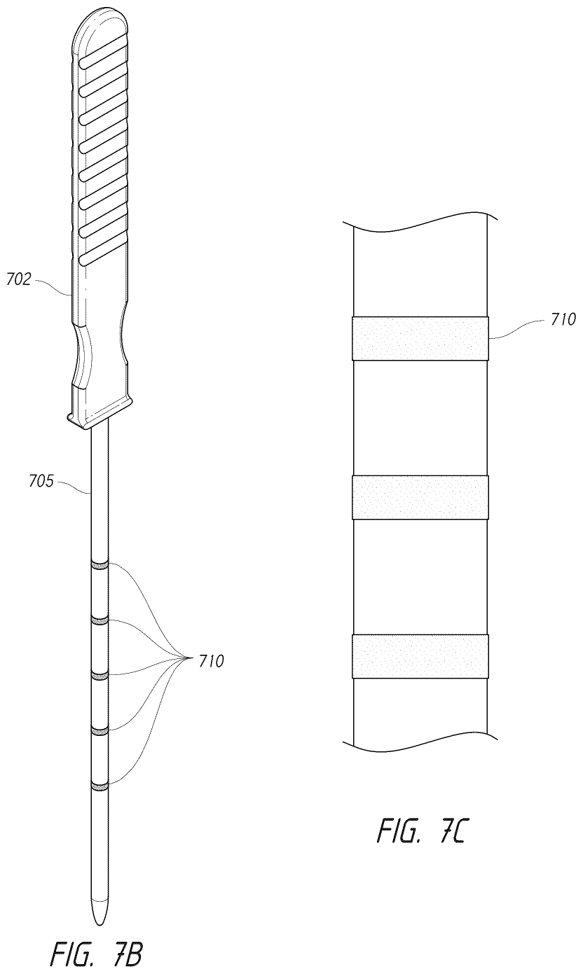

FIG. 7A is an isometric view of a multi-portal surgical system 100 in accordance with an embodiment of the disclosure. The system 100 is generally similar to the system 100 of FIG. 1A except as detailed below. The system 100 can include a depth guide 700 used to determine the size (e.g., depth) of incisions/cuts, position of anatomical features, and/or the like. As shown in FIG. 7A, the depth guide 700 can be inserted through the marking guide 150 and the incision 170A, and can be used to regulate the depth at which instruments (e.g., access instruments, surgical instruments, cannulas, etc.) penetrate the working space, thereby preventing or limiting injury or damage to non-targeted organs and tissues, such as nerve tissue.

FIG. 7B is an isometric view of the depth guide 700. The depth guide 700 includes a handle 702 and a shaft or body 705 extending distally therefrom. The body 705 is configured with depth markers or indicators 710 used to measure the depth of an incision and/or working space. FIG. 7C is an enlarged side view of three annular depth indicators 710 on the depth guide 700. The depth indicators 710 (e.g., etchings, rings, color coded annular markings, etc.) can be specific to the depths of the working space, proximity to anatomical structures, etc.

During a surgical procedure, the body 705 can be inserted through slots of the marking guide 150 (FIG. 1A-7A), the incisions 170 (FIGS. 1A-1C), and layers of tissue. The user can feel when the distal end of the depth guide 700 reaches uncut tissue (e.g., from the reaction force exerted by the uncut tissue on the depth guide 700). The user can then visually inspect the depth indicator 710 indicating the distance to the uncut tissue. The depth indicator 710 can be made of radiopaque material for visualization using fluoroscopy. The depth guide 700 can be used multiple times in a procedure to measure depths of different working spaces or changing depths of the same working space. For example, the depth guide 700 can be used to measure the depth to bony tissue and can be used any number of times based on, for example, the instrument being inserted, surgical steps to be performed, etc.

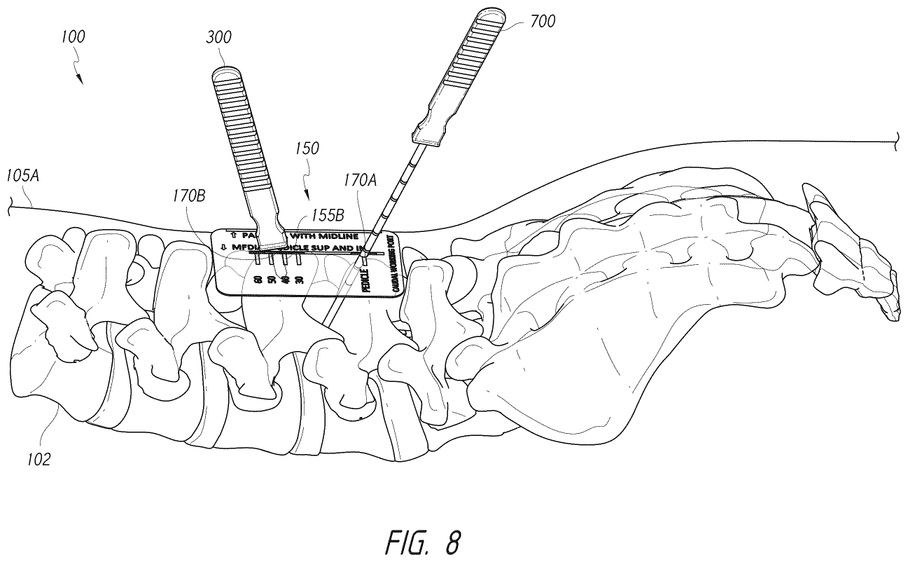

FIG. 8 shows components of the multi-portal surgical system 100 in accordance with an embodiment of the disclosure. The system 100 can include the marking guide 150 (as described in FIG. 1A-2D), the depth guide 700 (as described in FIG. 7A-7C), and the expandable scalpel 300 (as described in FIGS. 3A-3C). The scalpel 300 is positioned in the elongated slot 171 at an incision opening 155B of the marking guide 150. The expandable scalpel 300 can be used to create an incision 170B, as described in more detail with reference to FIG. 1A. Although not entirely illustrated in FIG. 8, the same process of clearing the workspace done in FIGS. 1A-7A can be done via the incision 170B. In some embodiments, the workspace cleared through the incision 170B is a visualization space for a visualization instrument (e.g., endoscopes, etc.) to be inserted.

FIGS. 9A and 9B show a component of the multi-portal surgical system 100 in accordance with an embodiment of the disclosure. A tissue dissector 900 (also referred to herein as “the paraspinal tissue dissector 900”) can include an elongate paraspinal tissue insertion body having a handle portion 902 and a surgical blade portion 905, a tissue dissection arm 950, and a trigger or lever 925 usable to create a larger working space in a patient. The lever 925 can be actuated to move the tissue dissection arm 950 from an undeployed position (FIG. 9A) to a deployed position (FIG. 9B) one or more times to split or separate additional tissue to create or enlarge a working space for instruments (surgical instrument, cannulas, combinations thereof, or the like).

FIG. 9C is an isometric view of the tissue dissector 900 in accordance with an embodiment of the disclosure. The handle portion 902 can be used to hold and/or grip the tissue dissector 900. The handle 902 can include one or more openings or through holes 930 configured to allow a user's fingers to fit inside or through the handle 902. The handle 902 can be made, in whole or in part, of one or more polymers, composites, plastics, metals, or materials and can include one more opening matching the openings 930. In some embodiments, the openings 930 can be lined with a compressible material to provide cushioning of the user's fingers during the surgical procedure. The surgical blade portion 905 and/or the tissue dissection arm 950 can be made from, for example, metals (e.g., steel, titanium, aluminum), polymers, ceramics, composites, or other biocompatible and sterilizable material.

FIG. 9D is a cross-sectional view of the tissue dissector 900 in accordance with an embodiment of the disclosure. The tissue dissector 900 can additionally include a link or connector 915 rotatably coupled to the lever 925 by one or more couplers 940 (e.g., hinges, pivots, joints, etc.). Sliders or pins 912, 914, 916 can be configured to move along slots 922, 924, 926, respectively, of the connector 915 when a force (e.g., by squeezing) is applied to the lever 925. In particular, the slot 926 is diagonally oriented such that as the connector 915 moves upward (e.g., toward the handle portion of the tissue dissector 900) in response to actuation of the lever 925, the pin 916, which is fixedly coupled to the arm 950 (thus also referred to herein as “the arm pin 916”), moves towards the right in FIG. 9D, thereby rotating and deploying the tissue dissection arm 950. In some embodiments, the tissue dissector 900 can further include a biasing element 927. The biasing element 927 can bias the lever 925 to move the tissue dissection arm 950 to the undeployed position (FIG. 9A). The force applied to the lever 925 can overcome biasing provided by the biasing element 927 to deploy the tissue dissection arm 950.

FIG. 10 is a side view of a multi-portal surgical system 100 in accordance with an embodiment of the disclosure. The system 100 is generally similar to the system 100 of FIG. 1A except as detailed below. Instruments 1020, 1050 can be angled toward each other while maintaining a minimum distance of separation. The instrument 1050 can be a working instrument 1050, and the instrument 1020 can be a visualization instrument 1020. One or more cannulas (not illustrated) can also be inserted into the incision to assist with insertion of the instruments 1020, 1050. The instruments 1020, 1050 can be moved distally and/or laterally in the working space and the visualization space, which can be positioned in incisions, cannulas, endoscopic ports, etc. to access a relatively large working space along the patient's spine. The distal portions of the respective instruments 1020, 1050 can be moved laterally, vertically, or other direction inside the working space.

In some procedures, the working instrument 1050 can be an access instrument visualized using endoscopic techniques, as discussed herein. For example, the working instrument 1050 can be used to remove additional tissue (e.g., intervertebral disc, tissue contributing to stenosis, etc.), form access paths to implantation sites, prepare an implantation site by, for example, moving organs or tissue (e.g., moving nerve tissue), prepare vertebral bodies (e.g., roughening or shaping vertebral endplates), and/or the like. The working instrument 1050 can also include a distraction instrument (e.g., one or more dilators) can be delivered through to distract adjacent vertebrae, thereby enlarging the intervertebral space. The working instrument 1050 can deliver an interbody implant through the working space, and/or through a cannula (e.g., a split cannula, a non-split tubular cannula, etc.), and into the enlarged intervertebral space.

In some procedures, the visualization instrument 1020 can be a low-profile fiber-optic endoscope positioned directly through an incision, an endoscopic port, and/or the like. The visualization instrument 1020 can include one or more endoscopes having, without limitation, fiber optics (e.g., optical fibers), lenses, imaging devices, working lumens, light source controls, and/or the like for direct viewing or viewing via a display 162 (e.g., an electronic screen, a monitor, etc.). In some embodiments, the visualization instrument 1020 can include a lumen through which fluid flows to irrigate the surgical site. For example, saline, or another suitable liquid, can be pumped through the visualization instrument 1020 to remove tissue (e.g., loose tissue, bone dust, etc.) or other material impairing visualization. The visualization instrument 1020 can also include one or more lumens (e.g., irrigation return lumens, vacuum lumens, etc.) through which the irrigation liquid can be withdrawn.

In some embodiments, the visualization instrument 1020 can be rod-lens endoscopes with an outer diameter equal to or smaller than about 2 mm, 3 mm, 4 mm, 5 mm, 6 mm, 8 mm, or 10 mm, and a length equal to or shorter than about 15 cm, 20 cm, 30 cm, or 40 cm. The visualization instrument 1020 can also have integrated irrigation features (e.g., valves, flow control buttons, fluid lumens, return lumens), connectors (e.g., electrical connectors, fluidic connectors, etc.), access ports (e.g., access ports connected to lumens (e.g., lumens through which instruments can pass)), and/or the like. In embodiments with an angled lens, the visualization instrument can have approximately 0-degree, 10-degree, 15-degree, 30-degree, or 45-degree lens angles, which are toward a light source. In other angled lens embodiments, the visualization instrument can have an approximately 15-degree, 30-degree, or 45-degree lens angled away from a light source. The angle of the lens can be selected based on the area to be viewed. In some posterior or lateral spinal procedures, a 0-degree lens can provide a wide-angle view suitable for viewing nerve roots, the spinal cord, and intervertebral space. A 30- or 45-degree lens endoscope angled toward the light source can be used to provide an angled view toward, for example, the spine or midsagittal plane to view, for example, the spinous processes, spinal cord, and/or central regions of the intervertebral space. A 30- or 45-degree lens endoscope angled away from the light source can be used to provide an angled view toward the lateral features of the spine, such as nerve roots at the neural foramen, side regions of the intervertebral space, and/or the like.

The visualization instrument 1020 can illuminate the body cavity and enable high-resolution video visualization. A light source (e.g., a laser, light-emitting diode, etc.) located near or at the proximal end of the fiber optics can be used to transmit light to the distal end and provide illuminating light. This enables a surgeon to safely navigate into the subject's body and to illuminate specific body anatomy to view vertebral spacing, vertebral structures, nerves, bony buildup (e.g., buildup that could be irritating and pressing against nerves contributing to nerve compression), etc. In some embodiments, visualization optics for vision and illumination are included within the distal tip of the visualization instrument 1020. The configuration and functionality of the visualization instrument 1020 can be selected based on the desired field of view, viewing resolution, pan/zoom functionality, and/or the like. Irrigation techniques, visualization devices, instruments, cannulas, and visualization and surgical techniques are discussed in U.S. application Ser. No. 17/902,685 and U.S. application Ser. No. 16/687,520, which are incorporated by reference in their entireties.

To position the instruments 1020, 1050, the instruments can be inserted into entrances (e.g., incisions formed using scalpels) formed in the subject's skin, as described in more detail with reference to FIGS. 1A-9D. A multi-portal instrument holder (not illustrated) can be adjusted to hold the instruments 1020, 1050 at fixed or altered positions once the instruments 1020, 1050 are delivered to the vertebral space. The multi-portal instrument holder can also be used to hold one or more cannulas in fixed or altered positions while the instruments 1020, 1050 are inserted through the cannulas to the vertebral space.

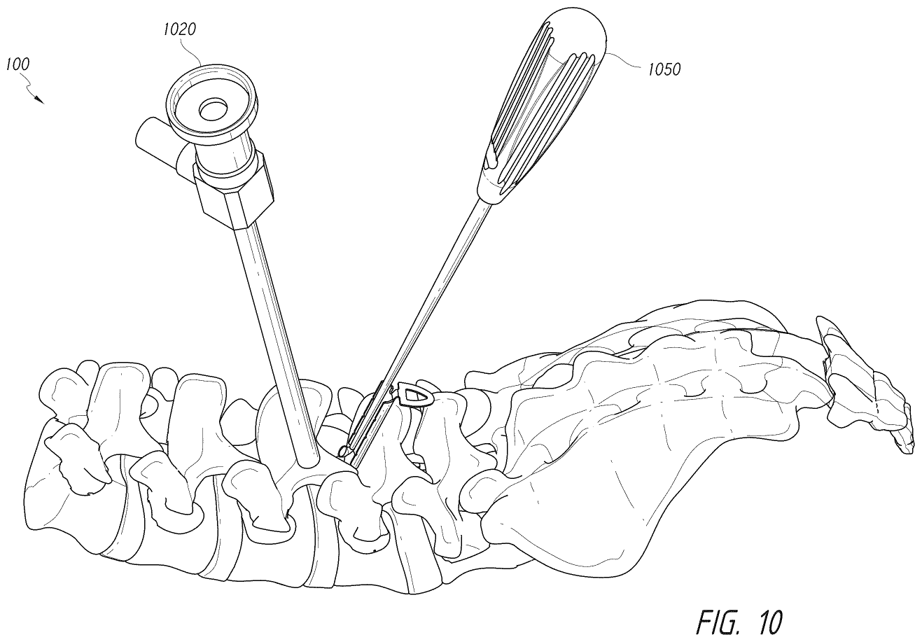

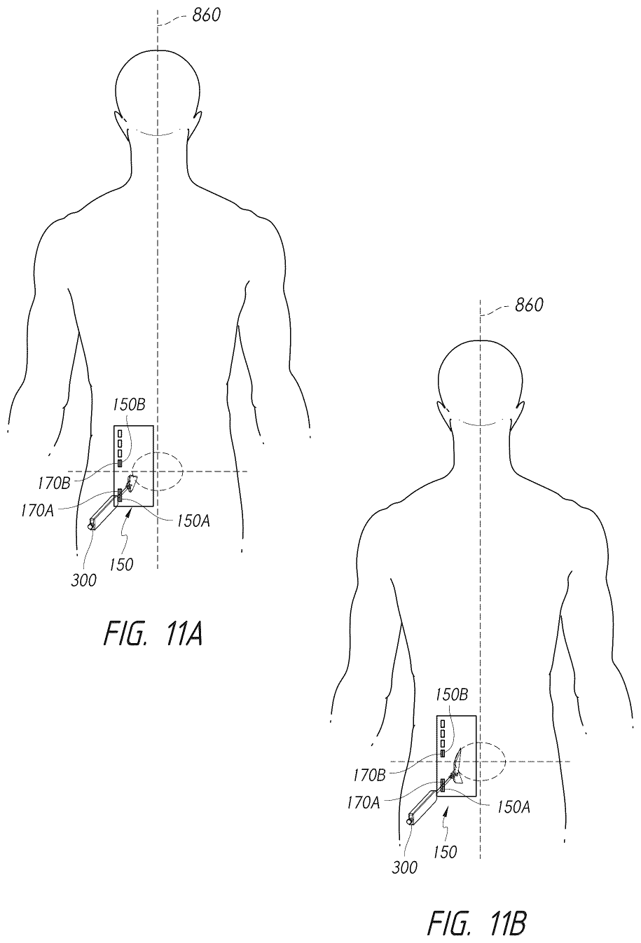

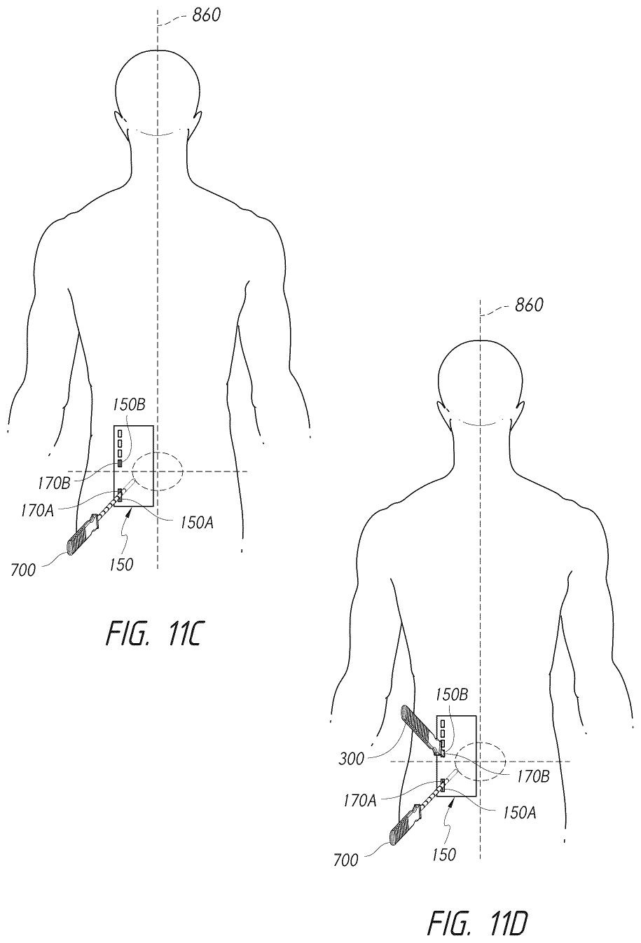

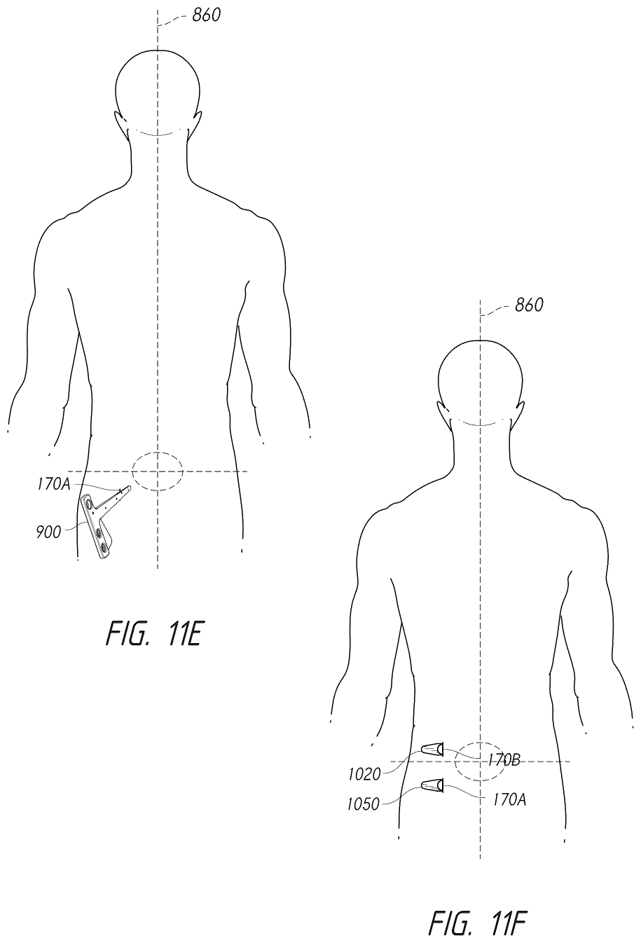

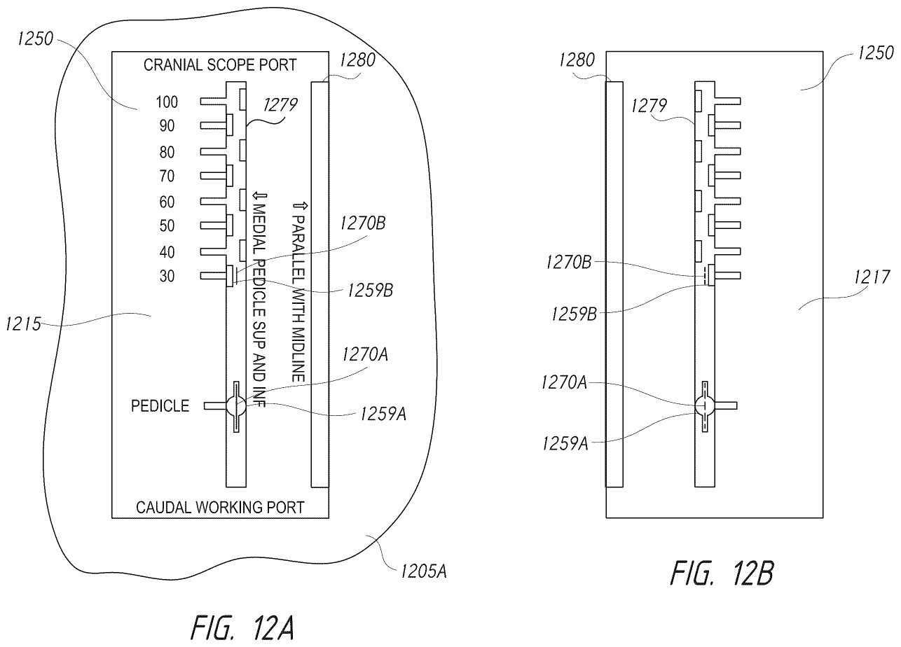

FIGS. 11A-11F illustrate surgical steps for performing spinal procedures in accordance with embodiments of the disclosure. FIG. 11A shows an incision 170A made using a slot 150A of a marking guide 150 for accessing the left side of a lumbar spine or the left side of the midsagittal plane 1160. The slot 150A can include an alignment feature 1159A used to align the incision 170A with features, such as one or more spinal markers, anatomical features (e.g., the left side pedicle, left side facet joint, spinous process, etc.), or the like. As further shown in FIG. 11A, the incision 170A is made using a scalpel 300 in an undeployed position. In some embodiments, the marking guide 150 can include multiple alignment features 1159A, 1159B. The multiple alignment features 1159A, 1159B can include incision markings, visualization features (e.g., radiopaque structures or elements sized and shaped to match or align with one or more features of the patient), planes, existing implants, and/or the like. For example, a user can position the scalpel 300 at the slot 150A using the alignment feature 1159A. An imaging device (e.g., a C-arm X-ray machine, camera, imager or camera of a robotic surgery system, etc.) can be used to confirm the relative position of the alignment feature 1159A along the patient, and the user creates the incision 170A. Marking guides can include any number of radiopaque alignment features. Example radiopaque alignment features are described in more detail with reference to the marking guides 1250, 1450, and 1550 of FIGS. 12A-15. FIG. 11B shows the scalpel 300 in a deployed position to alter and/or clear tissue. A cleared working path or space can follow a trajectory towards the vertebral space set using the marking guide 150.

FIG. 11C shows a depth guide 700 in the incision 170A to verify the depth, position, and trajectory of instruments to follow. FIG. 11D shows an incision 170B made using a slot 150B of the marking guide 150 for accessing another part the left side of a lumbar spine. As further shown in FIG. 11D, the incision 170B is made using the expandable scalpel 300 (as described in FIG. 11A).

Although not entirely illustrated in FIG. 11D, the same process of clearing and checking the workspace done in FIGS. 11A-11C can be done in the incision 170B. In some embodiments, the marking guide 150 can be repositioned and/or a second marking guide can be used to align and make the incision 170B based on, for example, imaging from the imaging device, visual inspection of the patient, or combinations thereof. The second marking guide can include additional slots and/or alignment features. For example, a user can replace the marking guide 150 with the second marking guide if the alignment features on the marking guide 150 do not align with a preferred spinal marker, if the user want to use different instruments, etc. Additionally or alternatively, a user can align the second marking guide vertically or horizontally at a proximal or distal end of the marking guide 150 to “extend” the slots and/or alignment features to the preferred spinal marker. The number and configuration of marking guides can be selected based on the surgical procedure. For example, different marking guides can be used at respective different levels of a multi-level spinal procedure. FIG. 11E shows the patient after the marking guide 150 has been removed, and the tissue dissector 900 positioned in the incision 170A. FIG. 11F shows cannulas 1022, 1055 positioned in the incisions 170A, 170B, respectively, for instrument insertion.

The instruments 1020, 1050 can be angled toward each other, as shown in FIG. 10, while maintaining a minimum distance of separation. In some embodiments, instruments are positioned within cannulas 1022, 1055 (FIG. 11F). Additional cannulas can be inserted into the subject to access other regions and/or provide alternative access paths. In some embodiments, a user can reposition the marking guide 150 and/or position a second marking guide onto the patient intraoperatively based on visualization from the visualization instrument 1020. For example, if visualization reveals misaligned instruments, obstacles or elements or unplanned anatomical changes, the user can align the marking guide 150, the second marking guide, or both the marking guide 150 and the second marking guide with one or more additional spinal markers to make an additional incision on the patient. The additional incision can reposition at least one of the instruments 1020, 1050. In an additional or alternative example, visualization can reveal that at least one of the instruments 1020, 1050 is not positioned along a preferred trajectory towards the working space. The user can align the marking guide 150, the second marking guide, or both the marking guide 150 and the second marking guide with one or more additional spinal markers to make an additional incision on the patient that realigns the at least one of the instruments 1020, 1050 along the preferred trajectory towards the working space. The marking guides can include visualization feature indicating instrument positions, trajectories, etc. The marking guides can be repositioned any number of times throughout the spinal procedure based on visualization from the visualization instrument 1020. Additionally or alternatively, the marking guides can be repositioned on the patient to maintain alignment of the instruments 1020, 1050 with the one or more spinal markers as the surgical procedure progresses.

Although the multi-portal surgical system 100 described in FIGS. 11A-11F includes the marking guide 150 on the human subject when forming incisions, the marking guide 150 can be removed from the patient at any time during the surgical procedure. For example, the marking guide 150 can be used to mark incisions using a surgical marking pen or skin marker (as described in FIG. 1A) and can be removed. The remainder of the steps (as described in FIGS. 11A-11F) can be performed without the marking guide 150. For example, access instruments described herein and/or other instruments used to clear the working space may be larger than the marking guide slots.

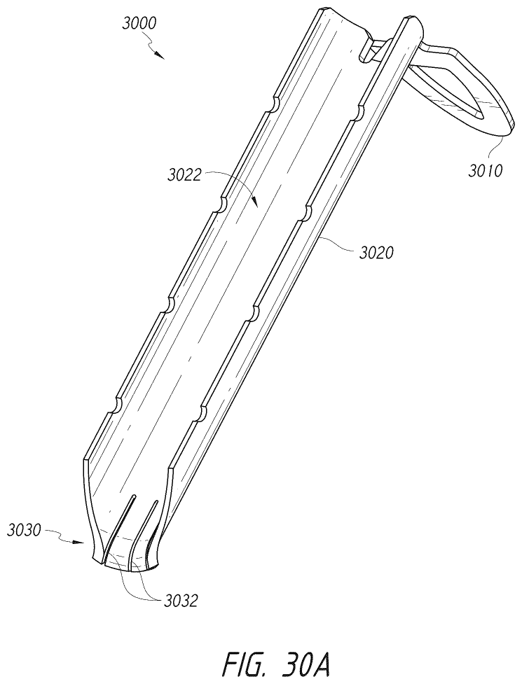

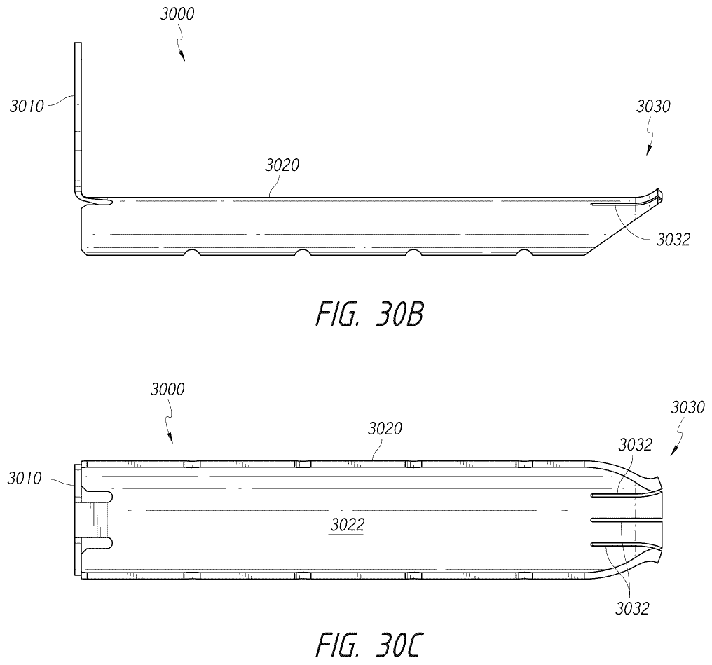

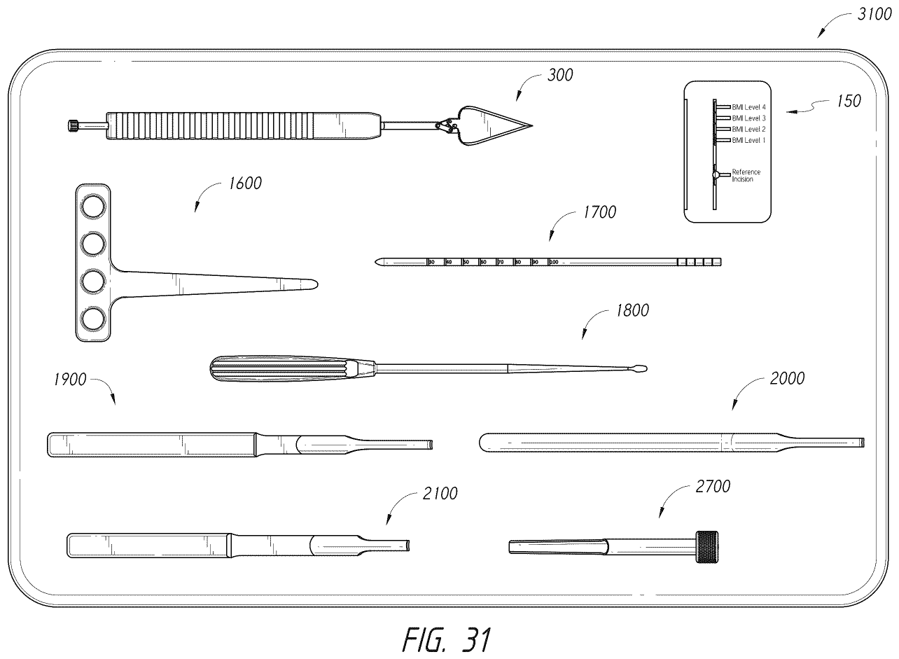

The components discussed herein can also be included in a surgical kit. The surgical kit can include one or more marking guides, expandable scalpels (single blade expandable scalpels, multi-blade expandable scalpels, etc.), a depth guide, and a tissue dissector. A physician can select appropriate marking guides and access tools based on the patient's size (e.g., BMI), the surgical procedure to be performed, and/or the like. The kit can further include a plurality of instruments (e.g., cannulas, debulking instruments, a reamer, etc.) Other instruments in the kit can include scalpels, dilators, rongeurs, or surgical instruments. The kit can also include components of, or an entire, the visualization instrument, the delivery or deployment instrument, and implantable devices. In some embodiments, the kit can also include cannulas (e.g., split cannulas, tubular cannulas, etc.), a set of ports, instrument holders to position the instruments (e.g., instruments 1020, 1050 of FIG. 10), and/or the like. An example kit is discussed in connection with FIG. 30.

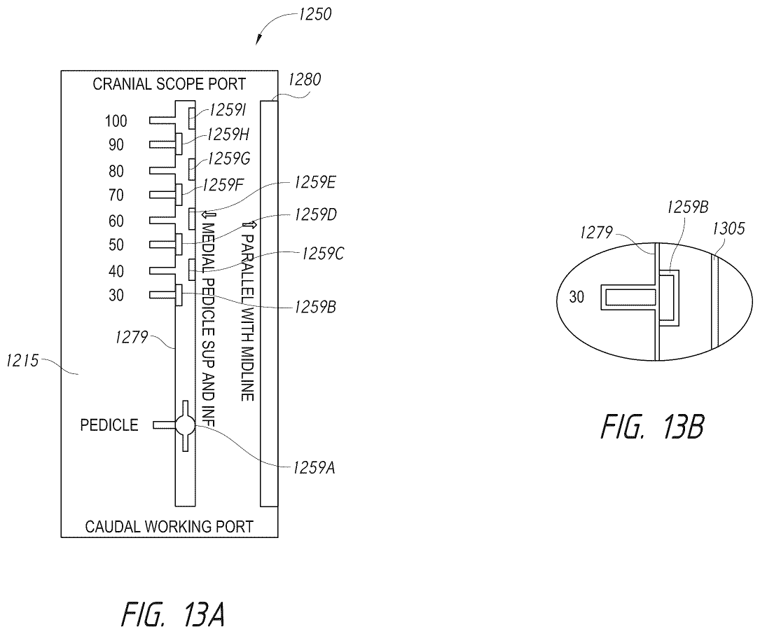

FIG. 12A is a top view of a marking guide 1250 positioned on a skin 1205 of a subject in accordance with an embodiment of the disclosure. FIG. 12B is a backside view of the marking guide 1250. The marking guide 1250 is generally similar to the marking guide 150 on the skin 105A, 105B (as shown in FIGS. 1B and 1C). The marking guide 1250 includes positioning features in the form of radiopaque alignment features. Referring to FIGS. 12A and 12B collectively now, the marking guide 1250 can include an elongated opening, slot, or port-alignment feature 1279 and a midline or plane alignment feature 1280. The elongated alignment feature 1279 and the plane alignment feature 1280 can be generally similar to the elongated slot 171 and the plane alignment feature 172 of FIGS. 1B-2B except the elongated alignment feature 1279 and the plane alignment feature 1280 can include radiopaque material (e.g., radiopaque material lining around one or more edges of the elongated alignment feature 1279 and the plane alignment feature 1280). The marking guide 1250 can also include multiple incision markings (e.g., incision markings 1259A-1259I discussed below in connection with FIG. 13A) corresponding to multiple incision sites. Each of the incision markings, for example the incision markings 1259A and 1259B, can have a corresponding marking label, legend (e.g., a triangulation legend, an instrument/scope legend, etc.), positioning labels (e.g., orientation labels, alignment labels, etc.), etc. on a top face 1215 of the marking guide 1250. As shown in FIGS. 12A and 12B, the corresponding marking label for the incision marking 1259A is illustrated as “PEDICLE” and for the incision marking 1259B is illustrated as BMI level “30” and the positioning labels illustrated as “PARALLEL WITH MIDLINE”, and orientation label illustrated as “CRANIAL” and “CAUDAL”.

The marking guide 1250, along with one or more access instruments, can be used to align, mark, and/or make initial incisions (as shown in FIGS. 1A-1C and 12A and 12B) and clear fascial tissue near the incision site (as shown in FIGS. 3A-3C). The marking guide 1250 can be removed, and one or more additional access instruments can be used to cut, spread, and/or remove additional tissue (as shown in FIGS. 9A-9D). In some embodiments, this creates a generally larger entrance for cannulas, surgical instruments, visualization instruments, and/or the like. The access instruments used to prepare a working path and/or space are discussed in more detail with reference to FIGS. 3A-10. Although not entirely illustrated in FIGS. 12A and 12B, the same process of clearing a working space done in FIGS. 1A-7A can be done via the incisions 1270A and 1270B. In some embodiments, the workspace cleared through the incision 1270A is a working space for a working instrument (e.g., rongeurs, scrapers, etc.) to be inserted. In some embodiments, the workspace cleared through the incision 1270B is a visualization space for a visualization instrument (e.g., endoscopes, etc.) to be inserted.

Incisions 1270A and 1270B (“incisions 1270”) can be made adjacent to the incision marking 1259A (e.g., Pedicle) and the incision marking 1259B (e.g., BMI Level 30) and/or generally in the center of the elongated alignment feature 1279. Additionally or alternatively, the incisions 1270 can be made within the incision markings 1259. A user can make the incisions 1270 using a scalpel (e.g., the expandable scalpel 300 of FIG. 1A in the elongated slot 171). In some embodiments, the user can position the scalpel relative in the elongated alignment feature 1279 and to one or more of the incision markings 1259. An imaging device (e.g., fluoroscopy, MRI imaging, CT imaging, direct visualization, etc.) can confirm the position of the incision markings 1259 relative to one or more spinal markers. For example, the user can align the incision marking 1259A with the pedicle of a vertebra, confirm the position of the incision marking 1259A at the pedicle, and identify a first incision location adjacent the incision marking 1259A. The guide 1250 can be oriented such that the plane alignment feature 1280 is generally parallel with the patient's midline. As shown in FIG. 12A, the guide 1250 can include arrows and text to help with positioning. Once the guide 1250 is aligned with the patient, a physician can make the working port incision 1270A directly above the pedicle. The same process of aligning the marking guide 1250 and creating the incision 1270A can be done to make the visualization port incision 1270B. In some embodiments, the marking guide 1250 is punctured using one or more access instruments adjacent to at least one alignment feature to make the incisions 1270A and/or 1270B. Additionally or alternatively, the marking guide 1250 can include one or more openings or slots configured to fit one or more access instruments that create the incisions 1270A and 1270B.

FIG. 12B is a backside view of the marking guide 1250. The marking guide 1250 can include a bottom face 1217 that can be coupled (e.g., coupled via an adhesive, a pressure-sensitive adhesive, etc.) to the skin (e.g., skin 1205 of FIG. 12A) to reduce, inhibit, or limit movement of the marking guide 1250 when marking skin, cutting tissue, etc. For example, the marking guide 1250 can be adhered to the skin 1205 such that when a user creates the incisions 1270A and 1270B, the incision markings 1259A and 1259B stay in generally the same position on the skin 1205.

In some embodiments, the bottom face 1217 can also include radiopaque material (e.g., embedded metal, deposited metal, printed metal) defining the incision markings 1259A-1259I, the elongated alignment feature 1279, and/or the plane alignment feature 1280 such that the alignment features can be seen from below that the marking guide 1250. Additionally or alternatively, one or more additional radiopaque alignment features can be embedded or adhered to the bottom face 1217 of the marking guide 1250 to enhance visibility of one or more additional spinal markers or features of the patient. In some embodiments, the user can align at least one of the incision markings 1259A-1259I, the elongated alignment feature 1279, or the plane alignment feature 1280 with at least one feature of the patient before coupling the bottom face 1217 to the skin 1205. This can ensure that the marking guide 1250 is in generally the correct position prior to adhering the marking guide 1250 to the skin 1205. It can be advantageous to confirm the position of the marking guide 1250 using radiopaque alignment features because it allows a user to orient the marking guide 1250 relative to features prior to creating an incision in the patient, thereby preventing excess harm to patient, and reducing the time spent creating and/or positioning instruments within a working space and/or a visualization space. In addition, the radiopaque alignment features on the bottom face 1217 can allow the user to monitor the position of the marking guide 1250, and thereby, the instruments and the instrument trajectories, using imaging (e.g., fluoroscopy, etc.) throughout the surgical procedure.

FIG. 13A is a top view of the marking guide 1250 of FIGS. 12A and 12B. The marking guide 1250 is configured with the elongated alignment feature 1279 on the left side of the top face 1215. The incision markings or openings 1259A-1259I (“incision markings 1259”) can alternate from the left or the right side of the elongated alignment feature 1279. The marking guide 1250 can further include marking labels (e.g., the Pedicle, BMI levels, etc.) to the left of the elongated alignment feature 1279. The plane alignment feature 1280 can extend in a lengthwise direction on the marking guide 1250 to orient the marking guide 1250 generally parallel to a midsagittal plane and/or supraspinous ligament of the patient. In operation, the marking guide 1250 can be used to make incisions for accessing the right side of a lumbar spine. Although not explicitly shown in FIG. 13A, a mirrored version of the marking guide 1250, as discussed in more detail with reference to FIG. 2A, can be used for accessing the left side of a lumbar spine. The incision markings 1259, the elongated alignment feature 1279, and/or the plane alignment feature 1280 can be radiopaque alignment features. The radiopaque alignment features can be defined by radiopaque material within the marking guide 1250 and/or the radiopaque alignment features can be one or more of a window, slot, and/or slit of the marking guide 1250 surrounded by a radiopaque lining, as described in more detail with reference to FIG. 13B.

The radiopaque alignment features can be compatible with one or more visualization techniques (e.g., fluoroscopy, MRI imaging, CT imaging, direct visualization, etc.). For example, radiopaque material can define the incision markings 1259, the elongated alignment feature 1279, and/or the plane alignment feature 1280 within the marking guide 1250. Additionally or alternatively, the elongated alignment feature 1279, the incision markings 1259, and/or the plane alignment feature 1280 can be one or more slots lined with a radiopaque material. The marking guide 1250 can also include guides and/or anatomical reference elements that are generally similar to the guides 200A, 200B and the anatomical reference elements 210A, 210B (as shown in FIGS. 2C and 2D) except the guides 200A, 200B and the anatomical reference elements 210A, 210B are made of and/or lined with a radiopaque material. In some embodiments, the radiopaque alignment features can be geometrically congruent to a relevant anatomical feature along and/or within the spine. The number, configuration, position, and/or orientation of the radiopaque alignment features can be selected based on the desired alignment of the marking guide 1250 with anatomical elements, planes, implants, and/or the like. For example, the marking guide 1250 can include radiopaque alignment features that align superjacent or adjacent to a patient's sagittal plane, midline, underlying anatomical features, one or both spinous processes, and/or the like.

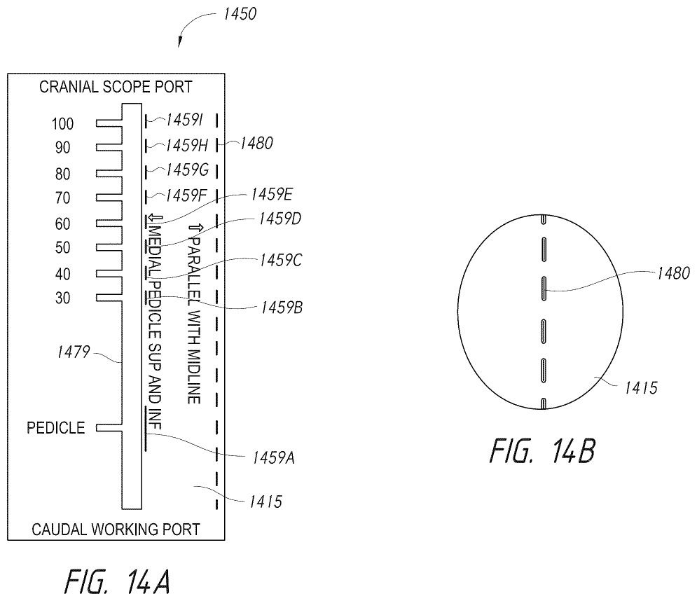

FIG. 13B shows the elongated alignment feature 1279 and the incision marking 1259B of FIG. 13A lined with or including a radiopaque material 1305. For example, the radiopaque material 1305 can line one or more edges of the elongated alignment feature 1279 and the incision markings 1259B. The radiopaque material 1305 can include metal, metal alloys, barium sulfate, and/or the like. In some embodiments, the marking guide 1250 is made of a polymer or plastic and the radiopaque material 1305 can prevent the user from cutting, cracking, and/or breaking the marking guide while creating incisions. Although not explicitly shown in FIG. 13B, the radiopaque material 1305 can also line the plane alignment feature 1280 of FIGS. 12A-13A in a similar manner. In some embodiments, the radiopaque material 1305 is embedded in the body of the guide 1250, as described in more detail with reference to FIGS. 14A and 14B.

FIG. 14A is a top view of a marking guide 1450 with embedded alignment features in accordance with an embodiment of the disclosure. The marking guide 1450 is generally similar to the marking guide 1250 of FIGS. 12A-13B except the marking guide 1450 includes radiopaque alignment features embedded into a top surface 1415 of the marking guide 1450. As shown in FIG. 14A, the marking guide 1450 includes an elongated alignment feature 1479 generally similar to the elongated alignment feature 1279 of FIGS. 12A-13B except that the elongated alignment feature 1479 does not include incision markings within the elongated alignment feature 1479. Rather, the incision markings 1459A-1249I (“incision markings 1459”) can be embedded into the top surface 1415 on the right side of the elongated alignment feature 1479. The marking guide 1450 can further include marking labels (e.g., the Pedicle, BMI levels, etc.) to the left of the elongated alignment feature 1479. A plane alignment feature 1480 can be embedded into the top surface 1415 of the marking guide 1450. The plane alignment feature 1480 can further extend in a lengthwise direction to orient the marking guide 1450 generally parallel to a midsagittal plane and/or supraspinous ligament of the patient.

The incision markings 1459 and/or the plane alignment feature 1480 can be radiopaque alignment features embedded into the top surface 1415. For example, the marking guide 1450 can include radiopaque alignment features that align superjacent or adjacent to a patient's sagittal plane, midline, underlying anatomical features, one or both spinous processes, and/or the like. The radiopaque alignment features can further include one or more radiopaque rods, strips, and/or adhesives super adjacent on the marking guide 1450 and/or adjacent a window, slot, and/or slit of the marking guide 1450. Although not explicitly shown in FIG. 14A, the elongated alignment feature 1479 can also include an additional radiopaque alignment feature embedded into the top surface 1415 that extends in a lengthwise direction on or along an entirety of either the right or left side of the elongated alignment feature 1479.

FIG. 14B shows the plane alignment feature 1480 embedded into the top surface 1415 of FIG. 14A. The plane alignment feature 1480 can be a radiopaque structure or element, for example, metal alloys, barium sulfate, and/or the like, that under imaging can orient the marking guide 1450 relative to the midsagittal plane of the patient. Although not explicitly shown in FIG. 14B, the elongated alignment feature 1479 and the incision markings 1459 can be radiopaque structures or elements embedded into the top surface 1415 of the marking guide 1450 in a similar manner to the plane alignment feature 1480. It is worth noting that any of the radiopaque alignment features depicted and/or described in FIGS. 14A and 14B as embedded into the top surface 1415 can also be adhered onto the top surface 1415. Additionally or alternatively, any of the radiopaque alignment features depicted and/or described in FIGS. 14A and 14B as embedded into the top surface 1415 can also be embedded and/or adhered to a bottom surface of the marking guide 1450.

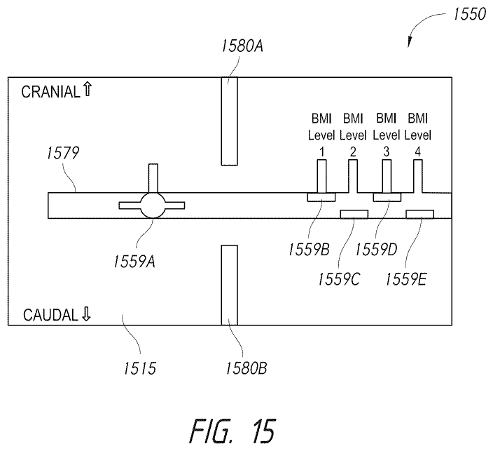

FIG. 15 is a top view of a marking guide 1550 with radiopaque alignment features in accordance with an embodiment of the disclosure. The marking guide 1550 can be generally similar to the marking guides 1250, 1450 of FIGS. 12A-14B except the marking guide 1550 has a horizontal orientation as opposed to a vertical orientation. The marking guide 1550 can include an elongated alignment feature 1579 that extends in a widthwise direction to orient the marking guide 1550 generally parallel to a transverse plane of the patient. The marking guide 1550 can also include incision markings or openings 1559A-1559E (“incision markings 1559”) that alternate from the upper side or lower side of the elongated alignment feature 1579. The marking guide 1550 can further include marking labels (e.g., Reference Incision, BMI levels, etc.) above the elongated alignment feature 1579. The plane alignment features 1580A and 1580B (“plane alignment features 1580”) can extend above and below the elongated alignment feature 1579, respectively, in a lengthwise direction to orient the marking guide 1550 generally parallel to a midsagittal plane of the patient. As shown in FIG. 15, the marking guide 1550 can include arrows and text to help with positioning. The incision markings 1559, the elongated alignment feature 1579, and/or the plane alignment feature 1580 can be radiopaque alignment features. In some embodiments, the radiopaque alignment features can be a window, slot, and/or slit of the marking guide 1550 surrounded by a radiopaque lining. Additionally or alternately, the radiopaque alignment features can be embedded or adhered to the top surface 1515 and/or bottom surface of the marking guide 1550.

It can be advantageous to use the horizontal marking guide 1550 for supraspinous implant procedures or bilateral spinal procedures where endoscopic or intervertebral access is required on both sides of the spine. In operation, the marking guide 1550 can be generally similar to the marking guides described herein in that a physician can select one or more of the incision markings 1559 to achieve target instrument trajectories relative to a working zone or target site. Additionally or alternatively, the radiopaque alignment features can allow the physician to make informed decisions in positioning and/or repositioning the marking guide 1550 based on image preoperatively and throughout the procedure. Although the radiopaque alignment features are described in more detail with reference to FIGS. 12A-15, any one of the marking guides described herein (e.g., the marking guide 150 of FIGS. 1A-3A, 7A, 8, and 11A-11D) can include one or more radiopaque alignment features.

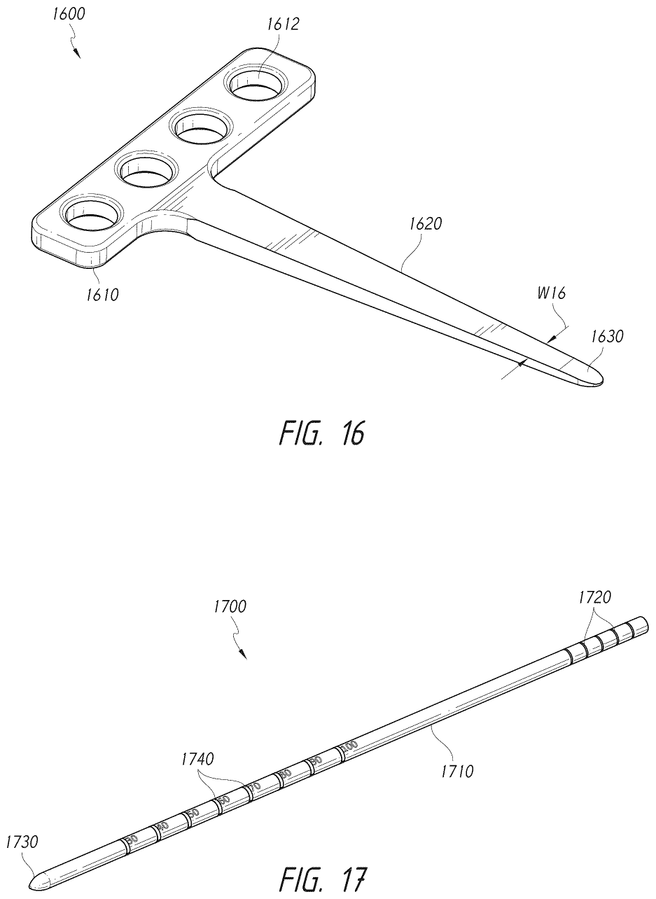

FIG. 16 is an isometric view of a muscle detacher 1600 in accordance with embodiments of the disclosure. The muscle detacher 1600 can include a handle portion 1610, an elongate body 1620, and a tapered tip 1630. The handle portion 1610 can include a plurality of openings 1612 sized and positioned to receive a user's fingers. For example, in the illustrated embodiment, the handle portion 1610 includes four openings 1612 sized and positioned to receive a user's index, middle, ring, and pinky fingers.

The elongate body 1620 can extend from the handle portion 1610 in a direction substantially perpendicular to the direction along which the openings 1612 are aligned. While the elongate body 1620 extends from a second one of the openings 1612 (e.g., the opening for receiving a user's middle finger) in the illustrated embodiment, the elongate body 1620 can extend from a different part of the handle portion 1610 in other embodiments. In some embodiments, the elongate body 1620 is tapered in a distal direction and has a variable width W16. The width W16 can generally match the size of the incision 170, the slots 151A-E and/or the incision openings 155A-E of the marking guide 150, and/or the like. For example, the width W16 can be between 2-10 mm (e.g., about 8 mm, 9 mm, 10 mm). The distal tip 1630 can have a flat and beveled and/or other suitable shape for pushing, cutting, or otherwise engaging and/or manipulating tissue.

The muscle detacher 1600 can be used to create a working space by inserting the distal tip 1630 and at least a portion of the elongate body 1620 through an incision (e.g., the incision 170) and moving the muscle detacher (e.g., in a scraping motion) to push apart subdermal tissue. In particular, the muscle detacher 1600 can be used in addition to or in lieu of the tissue dissector 900 of FIGS. 9A-9D. The openings 1612 can provide an ergonomic grip of the muscle detacher 1600, and the elongate body 1620 can be sized to be usable with, e.g., the marking guide 150.

FIG. 17 is an isometric view of a depth gauge 1700 in accordance with embodiments of the disclosure. The depth gauge 1700 can include an elongate body 1710 having one or more grooves 1720 at a proximal end portion thereof and/or a tapered distal end 1730. The grooves 1720 can facilitate a more secure handheld grip of the depth gauge 1700. The tapered distal end 1730 can facilitate insertion of the depth gauge 1700 into subdermal tissue. The elongate body 1710 can also have a plurality of depth markings 1740 positioned between the grooves 1720 and the tapered distal end 1730. The depth markings 1740 can include, for example, annular grooves, bands, notches, rings (e.g., colored rings, labelled rings, etc.) with numbers indicating the distance from the tapered distal end (e.g., in millimeters), or the like. In some embodiments, the depth markings 1740 and/or other portions of the depth gauge 1700 are radiopaque. For example, the depth markings 1740 can be radiopaque rings indicating distances between the respective rings and a distal tip of the depth gauge 1700. Other instruments, including instruments discussed in connection with FIGS. 3B-9D and 18A-29, can also include depth markings to assist with instrument positions.

The depth gauge 1700 can be used in substantially the same manner as the depth guide 700 of FIGS. 7A-7C. For example, after creating a workspace using the muscle detacher 1600 and/or other instrument, the depth gauge 1700 can be inserted therein to measure the size of the workspace created. The diameter of the elongate body 1710 can generally match or be smaller than the size of the incision 170, the slots 151A-E and/or the incision openings 155A-E of the marking guide 150, and/or the like. For example, the diameter can be between 2-10 mm (e.g., about 2 mm, 5 mm, 6 mm, 7 mm, 8 mm, 9 mm, 10 mm).

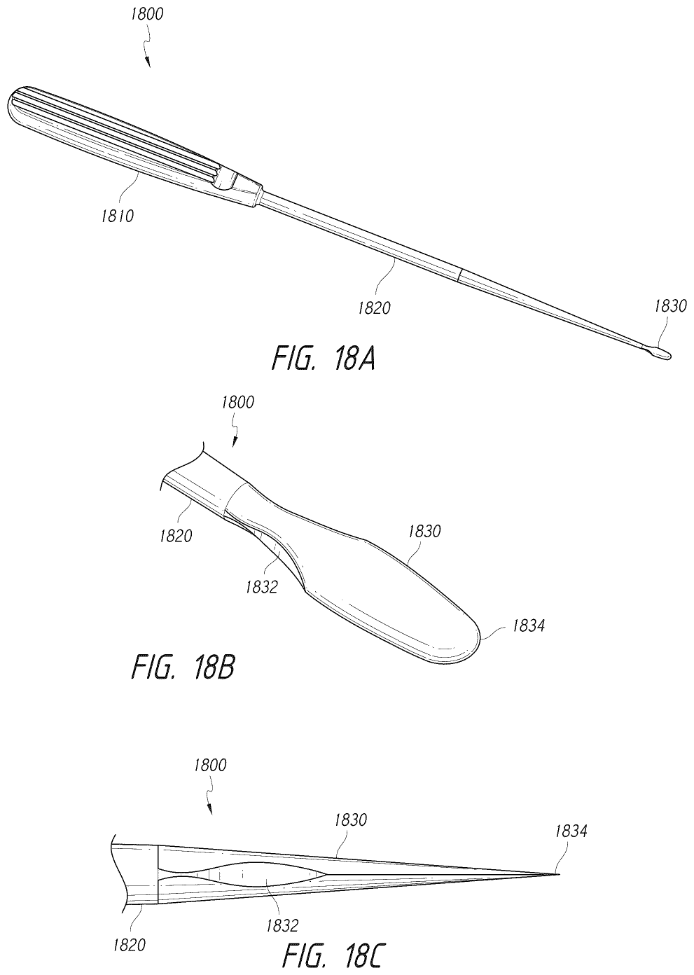

FIG. 18A is an isometric view of a spade knife 1800 in accordance with embodiments of the disclosure. The spade knife 1800 can include a handle portion 1810, an elongate body 1820 extending distally from the handle portion 1810, and a spade-shaped blade 1830 at a distal end of the elongate body 1820. The elongate body 1820 and the blade 1830 can be sized to be inserted through the slots 151A-E of the marking guide 150, the incision openings 155A-E of the marking guide 150, and/or in the incisions 170.

FIGS. 18B and 18C are enlarged isometric and side views, respectively, of the blade 1830 of the spade knife 1800. As shown, the blade 1830 can have a generally spade-shaped form factor with two proximal notches 1832 and a distal edge 1834. In particular, the distal edge 1834 can be generally arched (FIG. 18B) and the blade 1830 can be tapered in a distal direction when viewed from the side (FIG. 18C). The angle of the taper can be between 2-20 degrees or between 6-10 degrees, such as about 8 degrees. The generally flat, tapered, and curved form factor of the blade 1830 is expected to be advantageous. For example, the blade 1830 can be an atraumatic blade that is sufficiently sharp to cut and/or pierce through spinal discs, but not sharp enough to cut nerves (e.g., spinal cord, spinal ganglia, nerve roots along the spine, etc.) or otherwise pose a risk to other anatomical parts of the subject, which conventional scalpels and knives may be. In some embodiments, an edge of the blade 1830 can have radius equal to or greater than 50 nm, 75 nm, 100 nm, 150 nm, 200 nm, 300 nm, etc. and ranges encompassing such radius (e.g., 50 nm-100 nm, 75 nm-150 nm, 150 nm-300 nm, etc.). In some embodiments, an edge of the blade 1830 is a cutting edge for cutting relative hard or stiff tissue can have radius equal to or less than 50 nm, 75 nm, 100 nm, 150 nm, etc. In some embodiments, a kit can include a scalpel and a spade knife. The cutting edge of spade knife can be more dull than the scalpel such that a physician an access path using the scalpel and tissue near nerve tissue with the spade knife. Example ratios of a radius of the cutting edge of the spade knife to a radius of a scalpel (or a non-atraumatic knife) can be equal to or greater than 4, 5, 6, 7, 8, 9, or 10 or ranges encompassing such ratios (e.g., ranges of 4-8, 5-9, etc.). [Clark—please confirm/update dimensions and ratios.]

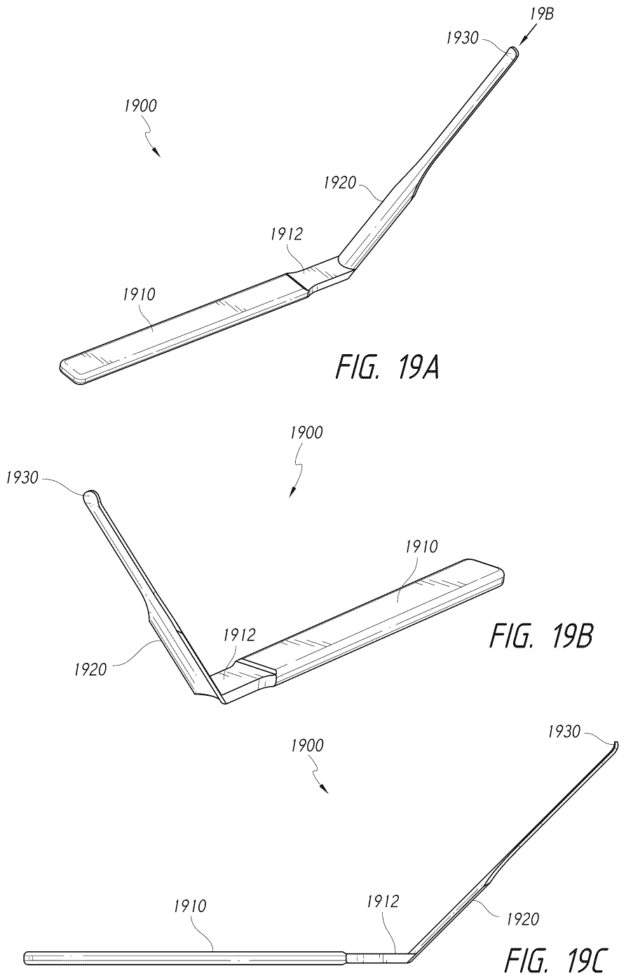

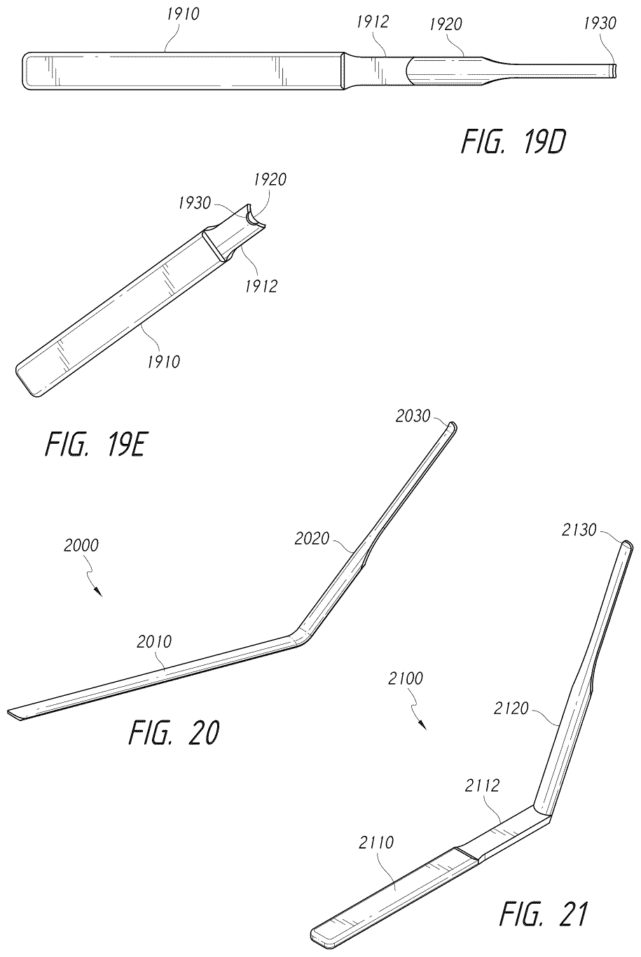

FIGS. 19A-19E are front isometric, rear isometric, side, top, and angled top views, respectively, of a root retractor 1900 in accordance with embodiments of the disclosure. In particular, FIG. 19E is a view of the root retractor 1900 in the direction of the arrow labeled 19E in FIG. 19A. Referring first to FIG. 19A, the root retractor 1900 can include a handle portion 1910, an insertion portion 1920, and an angled tip 1930. The handle portion 1910 can have a generally elongate form factor suitable for handheld gripping, and a neck portion 1912 with a reduced profile. The insertion portion 1920 can extend from the handle portion 1910 (e.g., from the neck portion 1912) at an angle, and can have a generally elongate form factor. The angle between the handle portion 1910 and the insertion portion 1920 can be between 90 degrees and 180 degrees, such as about 130 degrees, 135 degrees, or 140 degrees. In the illustrated embodiment, the insertion portion 1920 narrows in width along its length. The angled tip 1930 can be at the distal end of the insertion portion 1920, and can include a semicircular component oriented at an angle relative to the insertion portion 1920. The angle between the insertion portion 1920 and the angled tip 1930 can be between 90 degrees and 180 degrees, such as about 130 degrees, 135 degrees, or 140 degrees.

As best seen in FIGS. 19B and 19E, the insertion portion 1920 can have a generally curved or crescent-shaped cross-section, with the concave side facing away from the handle portion 1910. Thus, the insertion portion 1920 can have the shape of a portion of a cylinder, arcuate shape, etc.

In operation, a user (e.g., a surgeon) can grip the handle portion 1910 and insert the insertion portion 1920 through an incision in the subject. In some embodiments, a cannula (e.g., a split cannula) can be used to facilitate insertion of the insertion portion 1920 into the subject. Subsequently, the user can move the root retractor 1900 around to gently move and hold nerve roots (and/or tissue) aside, providing the user with better access and visibility to spinal structures while minimizing the risk of nerve damage. In particular, the angled tip 1930 can be shaped and sized to better hold the nerve roots (and/or tissue) and/or prevent tissue creep.

In some embodiments, once the root retractor 1900 is positioned in the subject (prior to, during, or after holding the nerve roots and/or tissue), the cannula used for guiding insertion of the insertion portion 1920 can be removed. Subsequently, another surgical instrument, such as an endoscope or a spinal implant delivery device, can be inserted through the same incision as the root retractor 1900 while the root retractor 1900 is still positioned in the patient. In particular, as described above with reference to FIG. 19B, the insertion portion 1920 can have a curved shape cross section, a crescent-shaped cross section or other cross-sectional shape such that the insertion portion 1920 can function as a split cannula for the insertion and use of the other surgical instrument.

FIG. 20 is an isometric view of a root retractor 2000 in accordance with embodiments of the disclosure. FIG. 21 is an isometric view of a root retractor 2100 in accordance with embodiments of the disclosure. It is appreciated that the root retractors 2000, 2100 may be generally similar to the root retractor 1900, and that similarly named and numbered elements described above are coupled and function similarly below. For example, the root retractor 2000 can include a handle portion 2010, an insertion portion 2020 extending from the handle portion 2010 at an angle, and an angled tip 2030 at the distal end of the insertion portion 2020. The root retractor 2100 can include a handle portion 2110, an insertion portion 2120 extending from the handle portion 2110 at an angle, and an angled tip 2130 at the distal end of the insertion portion 2120. The handle portion 2110 can include a neck portion 2112. Compared to the neck portion 1912, the neck portion 2112 can be longer.

In use, the root retractors 2000, 2100 can function similarly as the root retractor 1900. For example, a user can grip the handle portions 2010, 2110, insert the insertion portions 2020, 2120 through an incision into a subject, and leverage the angled tips 2030, 2130 to gently move and hold nerve roots and/or tissue to provide better access to a working space. Moreover, the insertion portions 2020, 2120, like the insertion portion 1920, can have a curved, crescent-shaped cross-section such that the insertion portions 2020, 2120 can serve as cannulas for subsequent surgical instruments inserted in the same incision as the root retractors 2000, 2100.

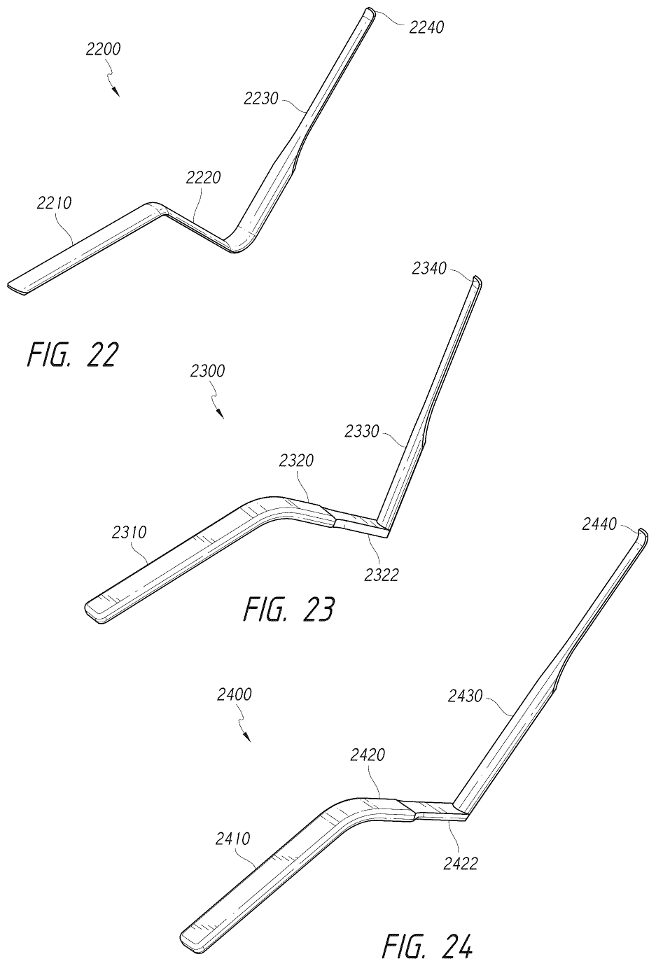

FIG. 22 is an isometric view of a root retractor bayonet 2200 in accordance with embodiments of the disclosure. The root retractor bayonet 2200 can include a handle portion 2210, an intermediate portion 2220, an insertion portion 2230, and an angled tip 2240. The intermediate portion 2220 can extend from the handle portion 2210 at a first angle (e.g., forming an angle therebetween of 90-180 degrees), the insertion portion 2230 can extend from the intermediate portion 2220 at a second angle (e.g., forming an angle therebetween of 70-180 degrees, such as 90 degrees), and the angled tip 2240 can extend from the insertion portion 2230 at a third angle (e.g., forming an angle therebetween of 90-180 degrees).

In use, the root retractor bayonet 2200 can function similarly as the root retractors 1900, 2000, 2100. For example, a user can grip the handle portion 2210, insert the insertion portion 2230 through an incision into a subject, and leverage the angled tip 2240 to atraumatically contact and/or gently move and hold nerve roots and/or tissue to provide better access to a working space and/or prevent tissue creep. Moreover, the insertion portion 2230, like the insertion portion 1920, can have a curved, crescent-shaped cross-section such that the insertion portion 2230 can serve as a cannula for subsequent surgical instruments inserted in the same incision as the root retractor bayonet 2200.

However, unlike the root retractors 1900, 2000, 2100, the root retractor bayonet 2200 includes the intermediate portion 2220 extending between the handle portion 2210 and the insertion portion 2230. In use, a user can position the intermediate portion 2220 against (e.g., in contact with) the skin of the subject, thereby better positioning the root retractor bayonet 2200 relative to the subject.

FIG. 23 is an isometric view of a root retractor bayonet 2300 in accordance with embodiments of the disclosure. FIG. 24 is an isometric view of a root retractor bayonet 2400 in accordance with embodiments of the disclosure. It is appreciated that the root retractor bayonets 2300, 2400 may be generally similar to the root retractor bayonet 2200, and that similarly named and numbered elements described above are coupled and function similarly below. For example, the root retractor bayonet 2300 can include a handle portion 2310, an intermediate portion 2320 extending from the handle portion 2310 at a first angle, an insertion portion 2330 extending from the intermediate portion 2320 at a second angle, and an angled tip 2340 extending from the insertion portion 2330 at a third angle. The intermediate portion 2320 can include a neck portion 2322. The root retractor bayonet 2400 can include a handle portion 2410, an intermediate portion 2420 extending from the handle portion 2410 at a first angle, an insertion portion 2430 extending from the intermediate portion 2420 at a second angle, and an angled tip 2440 extending from the insertion portion 2430 at a third angle. The intermediate portion 2420 can include a neck portion 2422.

In use, the root retractor bayonets 2300, 2400 can function similarly as the root retractor bayonet 2200. For example, a user can grip the handle portions 2310, 2410, insert the insertion portions 2330, 2430 through an incision into a subject, position the intermediate portions 2320, 2420 against the skin of the patient, and leverage the angled tips 2340, 2440 to gently move and hold nerve roots and/or tissue to provide better access to a working space. Moreover, the insertion portions 2330, 2430, like the insertion portion 2230, can have a curved, crescent-shaped cross-section such that the insertion portions 2330, 2430 can serve as cannulas for subsequent surgical instruments inserted in the same incision as the root retractor bayonets 2300, 2400.

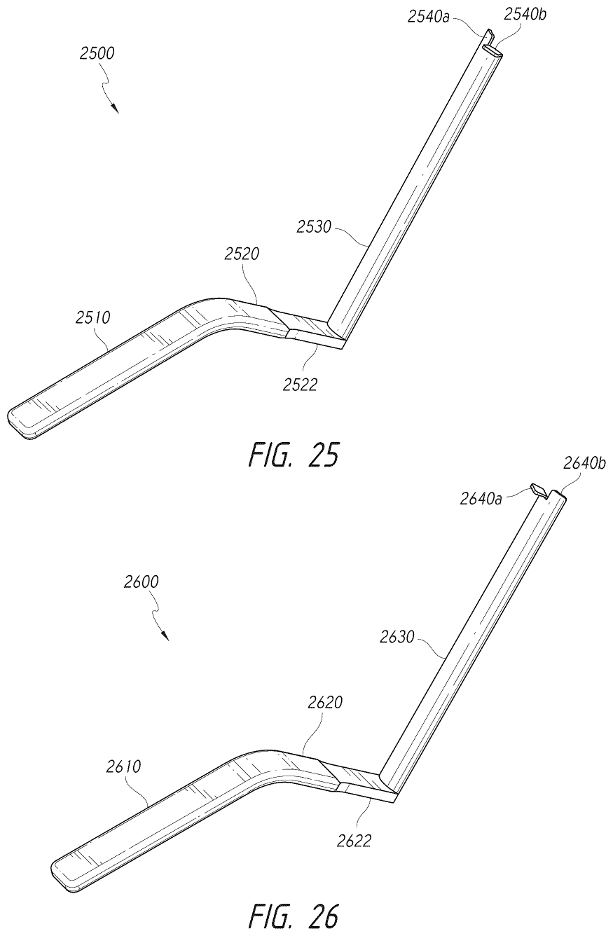

FIG. 25 is an isometric view of a fusion guide 2500 in accordance with embodiments of the disclosure. The fusion guide 2500 can include a handle portion 2510, an intermediate portion 2520, an insertion portion 2530, and a pair of tips 2540a, 2540b (collectively referred to as “the tips 2540”). The intermediate portion 2520 can include a neck portion 2522 and can extend from the handle portion 2510 at a first angle (e.g., forming an angle therebetween of 90-180 degrees), and the insertion portion 2530 can extend from the intermediate portion 2520 at a second angle (e.g., forming an angle therebetween of 70-180 degrees, such as 90 degrees). The first tip 2540a can extend generally parallel to the insertion portion 2530, and the second tip 2540b can extend from the insertion portion 2530 at a third angle (e.g., forming an angle therebetween of 70-180 degrees, such as 90 degrees). Therefore, the first tip 2540a can be a non-angled tip, and the second tip 2540b can be an angled tip.

FIG. 26 is an isometric view of a fusion guide 2600 in accordance with embodiments of the disclosure. The fusion guide 2600 can include a handle portion 2610, an intermediate portion 2620, an insertion portion 2630, and a pair of tips 2640a, 2640b (collectively referred to as “the tips 2640”). The intermediate portion 2620 can include a neck portion 2622 and can extend from the handle portion 2610 at a first angle (e.g., forming an angle therebetween of 90-180 degrees), and the insertion portion 2630 can extend from the intermediate portion 2620 at a second angle (e.g., forming an angle therebetween of 70-180 degrees, such as 90 degrees). The first tip 2640a can extend from the insertion portion 2630 at a third angle (e.g., forming an angle therebetween of 70-180 degrees, such as 90 degrees), and the second tip 2640b can extend generally parallel to the insertion portion 2630. Therefore, the first tip 2540a can be an angled tip, and the second tip 2540b can be a non-angled tip.

Referring to FIGS. 25 and 26 together, the fusion guides 2500, 2600 can be used to further expand a working space in a subject for, e.g., a spinal fusion procedure. For example, a user can grip the handle portions 2510, 2610, insert the insertion portions 2530, 2630 into an incision, position the intermediate portions 2520, 2620 against the skin of the subject, and leverage the tips 2540, 2640 to gently move and hold nerve roots and/or tissue to expand the working space for, e.g., implantation of a spinal implant (e.g., a cage implant). In particular, the insertion portions 2530, 2630 can each have a generally curved or crescent-shaped cross-section to serve as a split cannula that guides delivery of the spinal implant. Comparing the fusion guides 2500, 2600, the tips 2540, 2640 are mirrored versions of one another. Specifically, the first tip 2540a is angled in the fusion guide 2500, whereas the second tip 2640b is angled in the second fusion guide 2600. Therefore, a user can use the fusion guides 2500, 2600 in tandem by, e.g., using one at the left side of the implantation site and the other at the right side of the implantation site.

FIG. 27 is an isometric view of a scope cannula 2700 in accordance with embodiments of the disclosure. The scope cannula 2700 can include a gripping portion 2710, a set screw 2720, a tubular portion 2730, and an open channel portion 2740. The gripping portion 2710 can have a cylindrical form factor having a proximal opening (not shown) and a lateral aperture (not shown) for receiving the set screw 2720. Each of the gripping portion 2710 and the set screw 2720 can include textured (e.g., grooved) surfaces for facilitating handheld gripping by a user. The tubular portion 2730 can extend distally from the gripping portion 2710, and the open channel portion 2740 can extend distally from the tubular portion 2730. The open channel portion 2740 can have a generally flat end 2750. In some embodiments, the tubular portion 2730 and the open channel portion 2740 are integrally formed, while the gripping portion 2710 and the set screw 2720 can be separate components that may be assembled together.

In use, the scope cannula 2700 can be used as a guide or cannula for a surgical instrument, such as an endoscope or other visualization tool. For example, a user can first insert the open channel portion 2740 and/or the tubular portion 2730 into an incision in a subject, insert the endoscope into the proximal opening of the gripping portion 2710 such that the endoscope extends into the tubular portion 2730 and along the open channel portion 2740, and engage the set screw 2720 to fix the position of the endoscope relative to the scope cannula 2700. The open channel portion 2740 can serve as a split cannula that allows the user to manipulate (e.g., bend) the endoscope away therefrom as needed.

FIG. 28 is an isometric view of a scope cannula 2800 in accordance with embodiments of the disclosure. It is appreciated that the scope cannula 2800 may be generally similar to the scope cannula 2700, and that similarly named and numbered elements described above are coupled and function similarly below. For example, the scope cannula 2800 can include a gripping portion 2810, a set screw 2820, a tubular portion 2830, and an open channel portion 2840. However, unlike the scope cannula 2700, the scope cannula 2800 can additionally include a retractor portion 2850 at a distal end of the open channel portion 2840. In the illustrated embodiment, the retractor portion 2850 is generally arched with its concave side facing away from a longitudinal axis of the scope cannula 2800.

FIG. 29 is an isometric view of a scope cannula 2900 in accordance with embodiments of the disclosure. It is appreciated that the scope cannula 2900 may be generally similar to the scope cannula 2700, and that similarly named and numbered elements described above are coupled and function similarly below. For example, the scope cannula 2900 can include a gripping portion 2910, a set screw 2920, a tubular portion 2930, and an open channel portion 2940. However, unlike the scope cannula 2700, the scope cannula 2900 can additionally include a retractor portion 2950 at a distal end of the open channel portion 2940. In the illustrated embodiment, the retractor portion 2950 is generally flat and rectangular.

Referring to FIGS. 28 and 29 together, in use, in addition to serving as guides or cannulas for an endoscope or other instrument, the scope cannulas 2800, 2900 can be used to, via the retractor portions 2850, 2950, push, move, grip, manipulate, split, and/or hold tissue or other anatomical features. Specifically, the concave side of the retractor portion 2850 can be pressed against tissue to, e.g., keep said tissue out of the way for the endoscope. Therefore, the scope cannulas 2800, 2900 can be used to simultaneously guide the endoscope into a working space and maintain a clear access path for the endoscope to reach the working space. It is appreciated that the illustrated retractor portions 2850, 2950 are merely examples, and that in other embodiments, a scope cannula can include a retractor portion having other geometries and/or sizes. Also, a scope cannula can have tissue-manipulators and/or multiple retractor portions (e.g., grippers, fingers) at the distal end thereof.

In some embodiments, the retractor portions 2850, 2950 can be actuatable. For example, the retractor portions 2850, 2950 can be rotated about one or more axes, can flex (e.g., between arched and flat configurations), and/or the like. The retractor portions 2850, 2950 can be actuatable via a knob that can be rotated and/or moved, pull-wires, shape memory material phase transitioning, and/or the like. In embodiments in which the scope cannula includes multiple retractor portions, the multiple retractor portions may be actuatable together (e.g., using a single knob) or independently (e.g., using dedicated knobs). The number and configuration of tissue-manipulators, retractor portions, etc. can be selected based on the procedure.

FIGS. 30A-30C are isometric, side, and front views, respectively, of a cannula 3000 in accordance with embodiments of the disclosure. Referring to FIGS. 30A-30C together, the cannula 3000 can include a cannula body 3020 and a flange 3010 extending from a proximal end of the cannula body 3020 substantially perpendicular to the cannula body 3020. The cannula body 3020 can have an elongate form extending along an axis and defining an open channel 3022. Thus, the cannula 3000 can be a split cannula. The cannula body 3022 can include a distal end portion 3030 having one or more slots 3032 extending from the distal tip of and into the cannula body 3022 (three slots 3032 are shown in the illustrated embodiment). It is appreciated that other embodiments can include a different number and/or different lengths of the slots 3032. Moreover, as best seen in FIG. 30B, the distal end portion 3030 can be flared in a direction away from the open channel 3022 (e.g., in the same direction as the flange 3010).

In operation, the cannula 3000 can be used to guide insertion of a surgical instrument, a spinal implant, and/or the like into a subject (e.g., via an incision). In particular, the one or more slots 3032 can define tissue-manipulators and/or cantilevered portions of the distal end portion 3030. For example, the cantilevered portions can act as fingers, grippers, claws, and/or the like to hold tissue. The flared geometry of the distal end portion 3030 can further facilitate holding tissue. For example, a user (e.g., a surgeon) can manipulate the cannula 3000 such that the rear surface of the distal end portion 3030 presses against and thereby holds tissue away from a workspace in the subject while, e.g., an endoscope positioned in the open channel 3022 is used to visualize the cleared workspace. In some embodiments, the distal end portion 3030 can be actuatable in a similar manner as described above with respect to the retractor portions 2850, 2950.

It is appreciated that the various embodiments of distal tips or distal end portions, such as the tissue-manipulators, retractor portions 2850, 2950 of FIGS. 28 and 29 and the distal end portion 3030 including the one or more slots 3032 of FIG. 30, whether actuatable or not, can be included in other cannulas (and/or other instruments). For example, the retractor portions, the flared tips, and/or the slots described herein can be included in various open cannulas and closed cannulas, including those described in the patent references incorporated by reference herein in this application.

FIG. 31 is a plan view of a surgical kit 3100 in accordance with embodiments of the disclosure. Referring now to FIG. 31, the surgical kit 3100 can include the multi-portal marking guide 150 and a set of example instruments discussed in connection with FIGS. 1A-30. One or more of the instruments can be configured to be inserted through incisions formed using the marking guide 150. The marking guide 150 can include incision locators each with an incision length label with a length equal to or greater than maximum transverse dimensions of insertions portion the instruments. For example, the marking guide 150 can include a scope incision locator with an incision length label with a length equal to or great than a diameter of a distal portion of an endoscope (e.g., an endoscope of the kit, an endoscope available at a surgery suite, etc.). The marking guide 150 can include instrument incision locator with incision length labels with a length equal to or greater than a diameter of a distal portion (e.g., portion inserted into a patient) of one or more of the instruments. In some embodiments, two or more of instruments each have a distal portion with a maximum width less than a maximum length of one or more of the discrete incision locators.

The configuration and components of the kit can be selected based upon the procedure to be performed. In some embodiments, the kit's components can be configured to be specifically compatible with one another. For example, as discussed above, the muscle detacher 1600 and the spade knife 1800 can be sized to correspond to the slots 151A-E and/or the incision openings 155A-E of the marking guide 150. In some embodiments, the kit includes decompression instruments configured for performing a spinal decompression procedure. In some embodiments, the kit includes implantation site preparation instruments for performing a spinal procedure and may optionally include one or more implants. Moreover, one or more of the kit's components can be disposable and can be made from metal, polymer, ceramic, composite, or other biocompatible and sterilizable material. The kit can include a container for holding the components. The container can be a reusable or disposable tray (illustrated) or box. In operation, a user can select the marking guides, the access instruments, and any additional tools based on the location of the working space. In some procedures, marking guides can be selected based on the location (e.g., depth) of the tissue, anatomical structures surrounding access paths and/or targeted tissue, and/or configuration of instrument(s). Systems, components, and instruments disclosed herein can be disposable or reusable. For example, components of the kit can be disposable to prevent cross-contamination. As used herein, the term “disposable” when applied to a system or component (or combination of components), such as a marking guide, scalpel (e.g., expandable scalpel), cannula, port, dispenser, instrument, tool, or a distal tip or a head (e.g., a reamer head, a rongeur, etc.), is a broad term and generally means, without limitation, that the system or component in question is used a finite number of times and is then discarded. Some disposable components are used only once and are then discarded. In other embodiments, the components and instruments are non-disposable and can be used any number of times. In some kits, all of the components can be disposable to prevent cross-contamination. In some other kits, components (e.g., all or some of the components) can be reusable. The kit components can be reusable or disposable and configured to be used with one another.

### C. Examples

The present technology is illustrated, for example, according to various aspects described below as numbered examples (1, 2, 3, etc.) for convenience. These are provided as examples and do not limit the present technology. It is noted that any of the dependent examples may be combined in any combination, and placed into a respective independent example. The other examples can be presented in a similar manner.
1. A multi-portal surgical method comprising:
positioning a marking guide along a subject such that a plurality of incision openings of the marking guide are spaced apart in a selected direction relative to the subject;
aligning at least one alignment feature of the marking guide with at least one feature of the subject;
selecting a first incision opening and a second incision opening from the plurality of incision openings based on the alignment of the at least one alignment feature and a quantified characteristic of the subject; creating a first entrance along the subject using the first incision opening;
creating a second entrance along the subject using the second incision opening; and
viewing a distal end of a working instrument positioned in the first entrance using a visualization instrument positioned in the second entrance while the working instrument and the visualization instrument are both angled toward a working zone adjacent a subject's spine.
2. The multi-portal method of example 1, wherein the at least one alignment feature is configured to assist with positioning the marking guide superjacent to or adjacent to the at least one feature.
3. The multi-portal method of example 1 or example 2, wherein the feature is at least one of an anatomical element of a vertebra of the subject, an anatomical plane of the subject, or an existing vertebral implant in the subject.
4. The multi-portal method of any of examples 1-3, wherein the at least one alignment feature is at least one of a radiopaque element.
5. The multi-portal method of any of examples 1-4, wherein the at least one alignment feature is a radiopaque lining that surrounds at least one of the plurality of incision openings.
6. The multi-portal method of any of examples 1-5, wherein the at least one alignment feature is a radiopaque element embedded within the marking guide.
7. The multi-portal method of any of examples 1-6, further comprising adhering at least one radiopaque sticker onto a surface of the marking guide, wherein the radiopaque sticker is used to align the marking guide with the at least one feature of the subject.
8. The multi-portal method of any of examples 1-7, further comprising imaging the marking guide to confirm alignment of the at least one alignment feature with the at least one feature of the subject.
9. The multi-portal method of any of examples 1-8, wherein the plurality of incision openings of the marking guide are spaced apart in a superior-inferior direction relative to the subject.
10. The multi-portal method of any of examples 1-9, wherein the at least one alignment feature of the marking guide aligns with the at least one feature of the subject to position the marking guide in a superior-inferior direction relative to the subject for identifying a first incision location for the first entrance and a second incision location for the second entrance.
11. The multi-portal method of any of examples 1-10, wherein a superior end and an inferior end of the working zone are positioned alongside two adjacent vertebrae.
12. The multi-portal method of any of examples 1-11, wherein the plurality of incision openings of the marking guide are spaced apart in a medial-lateral direction relative to the subject when the marking guide rests on the subject.
13. The multi-portal method of any of examples 1-12, wherein the at least one alignment feature of the marking guide aligns with the at least one feature of the subject to position the marking guide in a medial-lateral direction relative to the subject for identifying a first incision location for the first entrance and a second incision location for the second entrance.
14. The multi-portal method of any of examples 1-13, further comprising selecting the marking guide from a set of marking guides based on the selected marking guide being configured for angling the working instrument and the visualization instrument into the working zone based on the quantified characteristic of the subject.
15. The multi-portal method of any of examples 1-14, wherein the working zone is a spinal-level working zone generally centered with respect to a disc space between two vertebrae.
16. The multi-portal method of any of examples 1-15, wherein the working zone is a spinal-level working zone generally centered with respect to a midsagittal plane of the subject between two features of a vertebra.
17. The multi-portal method of any of examples 1-16, wherein the first and second entrances are spaced apart based on a distance from a subject's skin to at least a portion of the working zone.
18. The multi-portal method of any of examples 1-17, wherein each of the incision openings has a corresponding body mass index label, and the quantified characteristic is a body mass index of the subject.
19. The multi-portal method of any of examples 1-18, further comprising:
applying a first skin marker at the first incision opening and a second skin marker at the second incision opening; and
forming the first entrance at the first skin marker and the second entrance at the second skin marker using at least one scalpel.
20. The multi-portal method of any of examples 1-19, further comprising:
inserting a scalpel through the first incision opening to form the first entrance; and
deploying a blade of the scalpel to cut tissue underlying a subject's skin to enlarge the working zone.
21. The multi-portal method of any of examples 1-20, further comprising:
after positioning the working instrument in the first entrance, altering tissue at the working zone using the working instrument; and
angling the visualization instrument toward the distal end of the working instrument to view the distal end of the working instrument altering of the tissue.
22. The multi-portal method of any of examples 1-21, further comprising using the first and second entrances to perform at least one of a decompression procedure, endoscopic spinal fusion procedure, or a bilateral spinal procedure.
23. The multi-portal method of any of examples 1-22, further comprising creating at least one of the first entrance and the second entrance using one or more access instruments.
24. The multi-portal method of example 23, wherein the one or more access instruments include at least one of an expandable scalpel, a depth guide, or a tissue dissector.
25. The multi-portal method of example 23 or example 24, wherein the one or more access instruments alter tissue by one or more of tearing, crushing, separating, cutting, debulking, breaking, fracturing, or removing of the tissue.
26. A marking guide for multi-portal procedures, the marking guide comprises a guide body with an elongated slot and one or more labeled openings used to align a scalpel and/or marking guide with a subject's body.
27. The marking guide of example 26, wherein the elongated slot is configured to receive one or more access instruments.
28. The marking guide of example 26 or example 27, wherein a radiopaque lining surrounds at least one of the elongated slots or the one or more labeled openings.
29. The marking guide of any of examples 26-28, wherein a radiopaque element is embedded to a surface of the guide body adjacent to at least one of the elongated slots or the one or more labeled openings.
30. The marking guide of any of examples 26-29, wherein a radiopaque element is adhered to a surface of the guide body adjacent to at least one of the elongated slots or the one or more labeled openings.
31. The marking guide of any of examples 26-30, wherein the marking guide further comprises one or more guides, wherein the one or more guides are configured to allow a marking tool to mark a subject's skin prior to incision.
32. The marking guide of any of examples 26-31, further comprising one or more anatomical reference elements configured to mate with one or more anatomical features such that the guide body is positioned between two identified anatomical elements of a subject's spine.
33. The marking guide of example 32, wherein the one or more anatomical reference elements are radiopaque.
34. A marking guide for multi-portal procedures, the marking guide comprises a guide body with multiple slots used to create incisions in a subject's skin for receiving multiple instruments for multi-instrument triangulation to a working space in the subject.
35. The marking guide of example 34, wherein the multiple slots are configured to receive one or more access instruments.
36. The marking guide of example 34 or example 35, wherein a radiopaque lining surrounds at least one of the multiple slots.
37. The marking guide of any of examples 34-36, wherein a radiopaque element is embedded to a surface of the guide body adjacent to at least one of the multiple slots.
38. The marking guide of any of examples 34-37, wherein a radiopaque element is adhered to a surface of the guide body adjacent to at least one of the multiple slots.
39. The marking guide of any of examples 34-38, wherein one of the slots is used to position a first incision along a first instrument trajectory toward the working space and another one of the slots is used to position a second incision along a second instrument trajectory toward the working space.
40. The marking guide of any of examples 34-39, further comprises one or more guides, wherein the one or more guides are configured to allow a marking tool to mark the subject's skin prior to incision.
41. The marking guide of any of examples 34-40, further comprising one or more anatomical reference elements configured to mate with one or more anatomical features such that the marking guide is positioned adjacent a vertebral level along a subject's spine.
42. The marking guide of example 41, wherein the one or more anatomical reference elements are radiopaque.
43. A marking guide for multi-portal procedures, the marking guide comprises a guide body with an elongated alignment feature and one or more labeled markers used to align a scalpel and/or marking guide with a subject's body.
44. The marking guide of example 43, wherein one of the labeled markers is used to position a first incision along a first instrument trajectory toward the working space and another one of the labeled markers is used to position a second incision along a second instrument trajectory toward the working space.
45. The marking guide of example 43 or example 44, wherein at least one of the elongated alignment feature or the one or more labeled markers are radiopaque elements embedded into a surface of the guide body.
46. The marking guide of any of examples 43-45, wherein at least one of the elongated alignment feature or the one or more labeled markers are adhered to a surface of the guide body.
47. The marking guide of any of examples 43-46, wherein the guide body further includes a plane alignment feature, and wherein the plane alignment feature is configured to align an anatomical plane of the subject.
48. A scalpel, comprising:
a body comprising:
   a first member portion;
   a spring; and
   a second member portion;
one or more blades configured to cut tissue; and
a trigger configured to cause the first member portion to compress the spring such that the spring pushes down on the second member portion and the second member portion deploys the one or more blades.
49. The scalpel of example 48, wherein the one or more blades are rotatable laterally outward away from the body.
50. The scalpel of example 48 or example 49, wherein the one or more blades has width or a length between 4 mm-16 mm.
51. The scalpel of any of examples 48-50, wherein the one or more blades are disposable.
52. A depth guide, comprising:
a handle; and
a body configured with depth indicators configured to measure a depth into a subject's body.
53. The depth guide of example 52, wherein the depth indicators are spaced apart based on a surgical procedure to be performed.
54. The depth guide of example 52 or example 53, wherein the body is configured for insertion into a working space in the subject's body or against one or more anatomical structures.
55. The depth guide of any of examples 52-54, wherein the depth indicators include one or more of etchings, rings, or color-coded annular markings.
56. A tissue dissector, comprising:
a handle;
a lever;
a cutting blade configured to cut tissue; and
a body having one or more slots and one or more sliders configured to move along respective ones of the slots when the lever is actuated to cause the cutting blade to rotate laterally outward.
57. The tissue dissector of example 56, wherein the handle includes one or more finger openings for gripping the handle without actuating the lever.
58. The tissue dissector of example 56 or example 57, wherein the lever includes a biasing element that biases the lever to move the cutting blade to an undeployed position.
59. A paraspinal tissue dissector, comprising:
a paraspinal tissue insertion body having a handle portion and a surgical blade portion extending distally from the handle portion,
a lever rotatably coupled to the paraspinal tissue insertion body at a first pivot in the handle portion of the paraspinal tissue insertion body,
a connector rotatably coupled to the lever at a second pivot spaced apart from the first pivot, wherein the connector extends along a length of the surgical blade portion of the paraspinal tissue insertion body, and wherein the connector includes a diagonally oriented slot, and
a tissue dissection arm rotatably coupled to the blade portion at a third pivot spaced apart from each of the first and second pivots, wherein the tissue dissection arm includes an arm pin positioned in the diagonally oriented slot of the connector,
wherein, in operation, actuation of the lever is configured to move the connector towards the handle portion, thereby causing (i) the arm pin to move along the diagonally oriented slot of the connector and (ii) the tissue dissection arm to rotate about the third pivot.
60. The tissue dissector of example 59, wherein the surgical blade portion includes one or more pins, wherein the connector further includes one or more elongated slots configured to receive corresponding ones of the one or more pins of the surgical blade portion, and wherein, in operation, actuation of the lever is configured to move the connector such that the one or more elongated slots of the connector move with respect to the corresponding ones of the one or more pins of the blade portion.
61. The tissue dissector of example 59 or example 60, further comprising a biasing element configured to bias the lever towards an unactuated position.
62. A surgical kit, comprising:
a marking guide having a plurality of elongated slots, wherein each of the plurality of elongated slots is configured to position an incision in a subject, and wherein the plurality of elongated slots are arranged such that different ones of the plurality of elongated slots correspond to different degrees of a physiological parameter; and
a plurality of access instruments, including:
   a muscle detacher having:
      a handle portion with a plurality of openings arranged along a first direction, wherein each of the plurality of openings is sized and positioned to receive a user's finger, and
      an elongate body extending from the handle portion in a second direction a substantially perpendicular to the first direction, wherein the elongate body is tapered in a distal direction along the second direction, and wherein a distal tip of the elongate body is substantially flat and beveled,
   a depth gauge having an elongate body with
      one or more grooves at a proximal end portion of the depth gauge, wherein the one or more grooves are configured to facilitate handheld grip of the proximal end portion,
      a tapered distal end configured to facilitate insertion of the depth gauge into the subject, and
      a plurality of depth markings positioned between the one or more grooves and the tapered distal end, and
   a spade knife having:
      a handle portion,
      an elongate body extending distally from the handle portion, and
      a spade-shaped blade at a distal end of the elongate body, wherein the spade-shaped blade is tapered at an angle between 6-10 degrees, and wherein the spade-shaped blade is an atraumatic blade,
wherein each of the plurality of access instruments is sized to fit at least partially into at least one of the plurality of elongated slots of the marking guide.
63. The surgical kit of example 62, wherein the physiological parameter is a body mass index (BMI) of the subject.
64. The surgical kit of example 62 or example 63, wherein a distal edge of the spade-shaped blade of the spade knife is arched, and wherein the spade-shaped blade is sharp enough to cut and/or pierce through spinal discs and not sharp enough to cut nerves.
65. The surgical kit of any of examples 62-64, further comprising a root retractor including:
a handle portion;
an insertion portion extending from the handle portion at an obtuse angle, wherein the insertion portion has a curved cross-section configured to guide insertion of a surgical instrument along the insertion portion; and
an angled tip at a distal end of the insertion portion.
66. The surgical kit of any of examples 62-65, further comprising a root retractor bayonet including:
a handle portion;
an intermediate portion extending from the handle portion at an obtuse angle;
an insertion portion extending from the intermediate portion at a substantially right angle, wherein the insertion portion has a curved cross-section configured to guide insertion of a surgical instrument along the insertion portion; and
an angled tip at a distal end of the insertion portion.
67. The surgical kit of any of examples 62-66, further comprising a fusion guide including:
a handle portion;
an intermediate portion extending from the handle portion at an obtuse angle;
an insertion portion extending from the intermediate portion at a substantially right angle;
an angled tip at a distal end of the insertion portion; and
a non-angled tip at a distal end of the insertion portion and positioned lateral to the angled tip.
68. The surgical kit of any of examples 62-66, further comprising a pair of fusion guides including:
a first fusion guide having:
   a first handle portion,
   a first intermediate portion extending from the first handle portion at an obtuse angle,
   a first insertion portion extending from the first intermediate portion at a substantially right angle,
   a first angled tip at a distal end of the first insertion portion, and
   a first non-angled tip at a distal end of the first insertion portion and positioned lateral to the first angled tip; and
a second fusion guide having:
   a second handle portion,
   a second intermediate portion extending from the second handle portion at an obtuse angle,
   a second insertion portion extending from the second intermediate portion at a substantially right angle,
   a second angled tip at a distal end of the second insertion portion, and
   a second non-angled tip at a distal end of the second insertion portion and positioned lateral to the second angled tip,
wherein (i) the first angled tip and the first non-angled tip are in a first arrangement and (ii) the second angled tip and the second non-angled tip are in a second arrangement that is mirrored relative to the first arrangement.
69. The surgical kit of any of examples 62-68, further comprising a scope cannula including:
a gripping portion having a first opening sized to receive an endoscope and a second opening oriented substantially perpendicular to the first opening;
a set screw sized to fit through the second opening of the gripping portion, wherein the set screw is configured to temporarily fix a position of the endoscope relative to the gripping portion;
a tubular portion extending in distally from the gripping portion; and
an open channel portion extending distally from the tubular portion.
70. The surgical kit of example 69, wherein the open channel has a substantially flat end.
71. The surgical kit of example 69, wherein the open channel has a substantially arched retractor portion at a distal end thereof, wherein a concave side of the arched retractor portion faces away from a longitudinal axis of the scope cannula.
72. The surgical kit of example 69, wherein the open channel has a substantially flat retractor portion at a distal end thereof.
73. A multi-portal surgical kit, comprising:
a multi-portal marking guide having a plurality of discrete incision locators including
   a first port incision locator indicating a first port location, and
   a set of second port incision locators each indicating a respective second port location, wherein the second port incision locators are arranged such that different ones of the second port incision locators correspond to different degrees of a physiological parameter; and
a set of instruments each configured to be inserted through one of
   a first incision at the first port location along a subject, or
   a second incision at one of the second port location along the subject.
74. The multi-portal surgical kit of example 73, wherein the marking guide includes a triangulation legend correlating the incision locators to the different degrees of the physiological parameter to triangulate toward a spine of subject both
an endoscope, which is position in the other one of the first incision or the second incision, and
one of the instruments positioned in the one of the first incision or the second incision.
75. The multi-portal surgical kit of example 74, wherein the triangulation legend includes body mass index values for respective ones of the second port incision locators.
76. The multi-portal surgical kit of any one of examples 73-75, wherein the multi-portal marking guide includes a plurality of physiological parameters each labeling a respective one of the incision locators.
77. The multi-portal surgical kit of any one of examples 73-76, wherein t
he multi-portal marking guide includes a planar body, and
the discrete incision locators each include an elongated through-hole or a slit in the planar body.
78. The multi-portal surgical kit of any one of examples 73-77, wherein one or more of the second port incision locators has an incision length label with a length equal to or greater than a maximum transverse dimension of an insertion portion of one of the instruments.
79. The multi-portal surgical kit of example 78, wherein the maximum transverse dimension is a diameter or width of the insertion portion.
80. The multi-portal surgical kit of any one of examples 73-79, wherein
the first port incision locator is a scope incision locator; and
one or more of the second port incision locators are working instrument port incision locators.
81. The multi-portal surgical kit of any one of examples 73-80, wherein
the first port incision locator is a working instrument port incision locator; and
one or more of the second port incision locators are scope port incision locators.
82. The multi-portal surgical kit of any one of examples 73-81, further including an endoscope, and the first port incision locator has an incision length label with a length equal to or great than a diameter of a distal portion of the endoscope.
83. The multi-portal surgical kit of any one of examples 73-82, wherein at least one of the second port incision locators includes an elongated slot configured to receive a scalpel blade of one of the instruments to form the second incision in a subject.
84. The multi-portal surgical kit of any one of examples 73-83, wherein two or more of the instruments each have distal portion with a maximum width with less than maximum length of one or more of the discrete incision locators.
85. The multi-portal surgical kit of any one of examples 73-84, wherein the set of instruments includes decompression instruments configured for performing a spinal decompression procedure on the subject.
86. The multi-portal surgical kit of any one of examples 73-85, wherein the set of instruments includes implantation site preparation instruments for spinal procedures.
87. The multi-portal surgical kit of any one of examples 73-86, wherein the set of instruments includes one or more of:
a muscle detacher having:
   a handle portion with a plurality of finger openings arranged along a first direction, and
   an elongate body extending from the handle portion in a second direction a substantially perpendicular to the first direction, wherein the elongate body is tapered in a distal direction along the second direction and includes a distal detacher tip configured for detaching muscle,
a depth gauge having an elongate body including
   one or more grooves at a proximal end portion of the depth gauge, wherein the one or more grooves are configured to facilitate handheld grip of the proximal end portion,
   a tapered distal end configured to facilitate insertion of the depth gauge into the subject, and
   a plurality of depth markings positioned between the one or more grooves and the tapered distal end,
a spade knife having:
   a handle portion,
   an elongate body extending distally from the handle portion, or
a spade-shaped blade at a distal end of the elongate body, wherein the spade-shaped blade is tapered at an angle between 6-10 degrees, and wherein the spade-shaped blade is an atraumatic blade.
88. The multi-portal surgical kit of example 87, wherein a distal edge of the spade-shaped blade of the spade knife is arcuate, and wherein the spade-shaped blade configured to cut and/or pierce through spinal discs and sufficient blunt to prevent cutting of nerves along subject's spine.
89. The multi-portal surgical kit of any one of examples 73-88, wherein each of the instruments is sized to fit at least partially into at least one of a plurality of elongated slots of the marking guide.
90. The multi-portal surgical kit of any one of examples 73-89, wherein the physiological parameter is a body mass index of the subject.
91. The multi-portal surgical kit of any one of examples 73-90, wherein at one least one of the instruments is a root retractor including:
a handle portion;
an insertion portion extending from the handle portion at an obtuse angle, wherein the insertion portion has a curved cross-section configured to guide insertion of a surgical instrument along the insertion portion; and
an angled tip at a distal end of the insertion portion.
92. The multi-portal surgical kit of any one of examples 73-91, further comprising a root retractor bayonet including:
a handle portion;
an intermediate portion extending from the handle portion at an obtuse angle;
an insertion portion extending from the intermediate portion at a substantially right angle, wherein the insertion portion has a curved cross-section configured to guide insertion of a surgical instrument along the insertion portion; and
an angled tip at a distal end of the insertion portion.
93. The surgical kit of any one of examples 73-92, further comprising a fusion guide including:
a handle portion;
an intermediate portion extending from the handle portion at an obtuse angle;
an insertion portion extending from the intermediate portion at a substantially right angle;
an angled tip at a distal end of the insertion portion; and
a non-angled tip at a distal end of the insertion portion and positioned lateral to the angled tip.
94. The multi-portal surgical kit of any one of examples 73-93, further comprising a pair of fusion guides including:
a first fusion guide having:
   a first handle portion,
   a first intermediate portion extending from the first handle portion at an obtuse angle,
   a first insertion portion extending from the first intermediate portion at a substantially right angle,
   a first angled tip at a distal end of the first insertion portion, and
   a first non-angled tip at a distal end of the first insertion portion and positioned lateral to the first angled tip; and
a second fusion guide having:
   a second handle portion,
   a second intermediate portion extending from the second handle portion at an obtuse angle,
   a second insertion portion extending from the second intermediate portion at a substantially right angle,
   a second angled tip at a distal end of the second insertion portion, and
   a second non-angled tip at a distal end of the second insertion portion and positioned lateral to the second angled tip,
wherein (i) the first angled tip and the first non-angled tip are in a first arrangement and (ii) the second angled tip and the second non-angled tip are in a second arrangement that is mirrored relative to the first arrangement.
95. The multi-portal surgical kit of any one of examples 73-94, further comprising a scope cannula including:
a gripping portion having a first opening sized to receive an endoscope and a second opening oriented substantially perpendicular to the first opening;
a set screw sized to fit through the second opening of the gripping portion, wherein the set screw is configured to temporarily fix a position of the endoscope relative to the gripping portion;
a tubular portion extending in distally from the gripping portion; and
an open channel portion extending distally from the tubular portion.

### D. Conclusion

The foregoing detailed description has set forth various embodiments of the devices and/or processes via the use of block diagrams, flowcharts, and/or examples.

Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one skilled in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal-bearing medium used to actually carry out the distribution. Examples of a signal-bearing medium include, but are not limited to, the following: a recordable type of medium such as a floppy disk, a hard disk drive, a CD, a DVD, a digital tape, a computer memory, etc.; and a transmission type medium such as a digital and/or an analog communication medium (e.g., a fiber-optic cable, a waveguide, a wired communications link, a wireless communication link, etc.).

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. Features from various systems, methods, and instruments can be combined with features disclosed in U.S. Provisional Patent Application No. 63/611,888; U.S. Provisional Patent Application No. 63/568,701, U.S. App. No. 63/611,913; U.S. application Ser. No. 15/793,950; U.S. application Ser. No. 17/902,685; U.S. application Ser. No. 18/335,737; U.S. application Ser. No. 18/464,949; U.S. application Ser. No. 18/470,140; U.S. application Ser. No. 18/764,784; U.S. Pat. Nos. 8,632,594; 9,308,099; 9,820,788; 8,632,594; 9,308,099; 9,820,788; 10,105,238; 10,201,431; 10,322,009; 10,898,340; 11,464,648; 11,950,770; PCT App. No. PCT/US20/49982; and PCT App. No. PCT/US22/21193, which are hereby incorporated by reference and made a part of this application. Variations of the implants are contemplated. The marking guides can include alignment features positioned to be aligned with anatomical features of the patient, such as a patient's spinous processes, supraspinous ligament, vertebral bodies, and/or the like. The alignment features can include marking labels and/or port and can be made, in whole or in part, of radiopaque materials to provide visualization using fluoroscopy to align the alignment features with underlying anatomical features. Example alignment features (e.g., BMI labels of 30, 40, 50, 60, pedicle alignment features or port, etc.).

Where the context permits, singular or plural terms may also include the plural or singular term, respectively. Moreover, unless the word “or”' is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

What is claimed is:

1. A multi-portal surgical method comprising:
positioning a marking guide along a subject such that a plurality of incision openings of the marking guide are spaced apart in a selected direction relative to the subject;
aligning at least one alignment feature of the marking guide with at least one feature of the subject;
selecting a first incision opening and a second incision opening from the plurality of incision openings based on (i) an alignment of the at least one alignment feature with the at least one feature of the subject and (ii) a quantified characteristic of the subject;
creating a first entrance along the subject using the first incision opening;
creating a second entrance along the subject using the second incision opening; and
viewing a distal end of a working instrument positioned in the first entrance using a visualization instrument positioned in the second entrance while the working instrument and the visualization instrument are both angled toward a working zone adjacent to a subject's spine.

2. The multi-portal method of claim 1, wherein the feature is at least one of an anatomical element of a vertebra of the subject, an anatomical plane of the subject, or an existing vertebral implant in the subject.

3. The multi-portal method of claim 1, wherein the at least one alignment feature is a radiopaque lining that surrounds at least one of the plurality of incision openings.

4. The multi-portal method of claim 1, further comprising adhering at least one radiopaque sticker onto a surface of the marking guide, wherein the radiopaque sticker is used to align the marking guide with the at least one feature of the subject.

5. The multi-portal method of claim 1, wherein the at least one alignment feature of the marking guide aligns with the at least one feature of the subject to position the marking guide in a superior-inferior direction relative to the subject for identifying a first incision location for the first entrance and a second incision location for the second entrance.

6. The multi-portal method of claim 1, further comprising selecting the marking guide from a set of marking guides based on the selected marking guide being configured for angling the working instrument and the visualization instrument into the working zone based on the quantified characteristic of the subject.

7. The multi-portal method of claim 1, wherein the working zone is a spinal-level working zone generally centered with respect to at least one of (i) a disc space between two vertebrae or (ii) a midsagittal plane of the subject between two features of a vertebra.

8. The multi-portal method of claim 1, wherein each of the incision openings has a corresponding body mass index label, and the quantified characteristic is a body mass index of the subject.

9. The multi-portal method of claim 1, further comprising:
applying a first skin marker at the first incision opening and a second skin marker at the second incision opening; and
forming the first entrance at the first skin marker and the second entrance at the second skin marker using at least one scalpel.

10. The multi-portal method of claim 1, further comprising:
inserting a scalpel through the first incision opening to form the first entrance; and
deploying a blade of the scalpel to cut tissue underlying a subject's skin to enlarge the working zone.

11. The multi-portal method of claim 1, further comprising:
after positioning the working instrument in the first entrance, altering tissue at the working zone using the working instrument; and
angling the visualization instrument toward the distal end of the working instrument to view the distal end of the working instrument altering of the tissue.

12. A paraspinal tissue dissector, comprising:
a paraspinal tissue insertion body having a handle portion and a surgical blade portion extending distally from the handle portion;
a lever rotatably coupled to the paraspinal tissue insertion body at a first pivot in the handle portion of the paraspinal tissue insertion body;
a connector rotatably coupled to the lever at a second pivot spaced apart from the first pivot, wherein the connector extends along a length of the surgical blade portion of the paraspinal tissue insertion body, and wherein the connector includes a diagonally oriented slot; and
a tissue dissection arm rotatably coupled to the blade portion at a third pivot spaced apart from each of the first and second pivots, wherein the tissue dissection arm includes an arm pin positioned in the diagonally oriented slot of the connector,
wherein, in operation, actuation of the lever is configured to move the connector towards the handle portion, thereby causing (i) the arm pin to move along the diagonally oriented slot of the connector and (ii) the tissue dissection arm to rotate about the third pivot.

13. The tissue dissector of claim 12, wherein the surgical blade portion includes one or more pins, wherein the connector further includes one or more elongated slots configured to receive corresponding ones of the one or more pins of the surgical blade portion, and wherein, in operation, actuation of the lever is configured to move the connector such that the one or more elongated slots of the connector move with respect to the corresponding ones of the one or more pins of the blade portion.

14. The tissue dissector of claim 12, further comprising a biasing element configured to bias the lever towards an unactuated position.

15. A multi-portal surgical kit, comprising:
a multi-portal marking guide having a plurality of discrete incision locators including
a first port incision locator indicating a first port location, and
a set of second port incision locators each indicating a respective second port location, wherein the second port incision locators are arranged such that different ones of the second port incision locators correspond to different degrees of a physiological parameter; and
a set of instruments each configured to be inserted through one of
a first incision at the first port location along a subject, or
a second incision at one of the second port location along the subject.

16. The multi-portal surgical kit of claim 15, wherein the marking guide includes a triangulation legend correlating the incision locators to the different degrees of the physiological parameter to triangulate toward a spine of subject both
an endoscope, which is position in the other one of the first incision or the second incision, and
one of the instruments positioned in the one of the first incision or the second incision.

17. The multi-portal surgical kit of claim 16, wherein the triangulation legend includes body mass index values for respective ones of the second port incision locators.

18. The multi-portal surgical kit of claim 15, wherein the multi-portal marking guide includes a plurality of physiological parameters each labeling a respective one of the incision locators.

19. The multi-portal surgical kit of claim 15, wherein
the multi-portal marking guide includes a planar body, and
the discrete incision locators each include an elongated through-hole or a slit in the planar body.

20. The multi-portal surgical kit of claim 15, wherein one or more of the second port incision locators has an incision length label with a length equal to or greater than a maximum transverse dimension of an insertion portion of one of the instruments.

21. The multi-portal surgical kit of claim 20, wherein the maximum transverse dimension is a diameter or width of the insertion portion.

22. The multi-portal surgical kit of claim 15, wherein
the first port incision locator is a scope incision locator; and
one or more of the second port incision locators are working instrument port incision locators.

23. The multi-portal surgical kit of claim 15, wherein
the first port incision locator is a working instrument port incision locator; and
one or more of the second port incision locators are scope port incision locators.

24. The multi-portal surgical kit of claim 15, further including an endoscope, and the first port incision locator has an incision length label with a length equal to or great than a diameter of a distal portion of the endoscope.

25. The multi-portal surgical kit of claim 15, wherein at least one of the second port incision locators includes an elongated slot configured to receive a scalpel blade of one of the instruments to form the second incision in a subject.

26. The multi-portal surgical kit of claim 15, wherein two or more of the instruments each have distal portion with a maximum width with less than maximum length of one or more of the discrete incision locators.

27. The multi-portal surgical kit of claim 15, wherein the set of instruments includes decompression instruments configured for performing a spinal decompression procedure on the subject.

28. The multi-portal surgical kit of claim 15, wherein the set of instruments includes implantation site preparation instruments for spinal procedures.

29. The multi-portal surgical kit of claim 15, wherein the set of instruments includes one or more of:
a muscle detacher having:
a handle portion with a plurality of finger openings arranged along a first direction, and
an elongate body extending from the handle portion in a second direction a substantially perpendicular to the first direction, wherein the elongate body is tapered in a distal direction along the second direction and includes a distal detacher tip configured for detaching muscle,
a depth gauge having an elongate body including
one or more grooves at a proximal end portion of the depth gauge, wherein the one or more grooves are configured to facilitate handheld grip of the proximal end portion,
a tapered distal end configured to facilitate insertion of the depth gauge into the subject, and
a plurality of depth markings positioned between the one or more grooves and the tapered distal end,
a spade knife having:
a handle portion,
an elongate body extending distally from the handle portion, or
a spade-shaped blade at a distal end of the elongate body, wherein the spade-shaped blade is tapered at an angle between 6-10 degrees, and wherein the spade-shaped blade is an atraumatic blade.

30. The multi-portal surgical kit of claim 29, wherein a distal edge of the spade-shaped blade of the spade knife is arcuate, and wherein the spade-shaped blade configured to cut and/or pierce through spinal discs and sufficient blunt to prevent cutting of nerves along subject's spine.

31. The multi-portal surgical kit of claim 15, wherein each of the instruments is sized to fit at least partially into at least one of a plurality of elongated slots of the marking guide.

32. The multi-portal surgical kit of claim 15, wherein the physiological parameter is a body mass index of the subject.

33. The multi-portal surgical kit of claim 15, wherein at one least one of the instruments is a root retractor including:
a handle portion;
an insertion portion extending from the handle portion at an obtuse angle, wherein the insertion portion has a curved cross-section configured to guide insertion of a surgical instrument along the insertion portion; and
an angled tip at a distal end of the insertion portion.

34. The multi-portal surgical kit of claim 15, further comprising a root retractor bayonet including:
a handle portion;
an intermediate portion extending from the handle portion at an obtuse angle;
an insertion portion extending from the intermediate portion at a substantially right angle, wherein the insertion portion has a curved cross-section configured to guide insertion of a surgical instrument along the insertion portion; and
an angled tip at a distal end of the insertion portion.

35. The surgical kit of claim 15, further comprising a fusion guide including:
a handle portion;
an intermediate portion extending from the handle portion at an obtuse angle;
an insertion portion extending from the intermediate portion at a substantially right angle;
an angled tip at a distal end of the insertion portion; and
a non-angled tip at a distal end of the insertion portion and positioned lateral to the angled tip.

36. The multi-portal surgical kit of claim 15, further comprising a pair of fusion guides including:
a first fusion guide having:
a first handle portion,
a first intermediate portion extending from the first handle portion at an obtuse angle,
a first insertion portion extending from the first intermediate portion at a substantially right angle,
a first angled tip at a distal end of the first insertion portion, and
a first non-angled tip at a distal end of the first insertion portion and positioned lateral to the first angled tip; and
a second fusion guide having:
a second handle portion,
a second intermediate portion extending from the second handle portion at an obtuse angle,
a second insertion portion extending from the second intermediate portion at a substantially right angle,
a second angled tip at a distal end of the second insertion portion, and
a second non-angled tip at a distal end of the second insertion portion and positioned lateral to the second angled tip,
wherein (i) the first angled tip and the first non-angled tip are in a first arrangement and (ii) the second angled tip and the second non-angled tip are in a second arrangement that is mirrored relative to the first arrangement.

37. The multi-portal surgical kit of claim 15, further comprising a scope cannula including:
a gripping portion having a first opening sized to receive an endoscope and a second opening oriented substantially perpendicular to the first opening;
a set screw sized to fit through the second opening of the gripping portion, wherein the set screw is configured to temporarily fix a position of the endoscope relative to the gripping portion;
a tubular portion extending in distally from the gripping portion; and
an open channel portion extending distally from the tubular portion.

38. A surgical kit, comprising:
a marking guide having a plurality of elongated slots, wherein each of the plurality of elongated slots is configured to position an incision in a subject, and wherein the plurality of elongated slots are arranged such that different ones of the plurality of elongated slots correspond to different degrees of a physiological parameter; and
a plurality of access instruments, including:
a muscle detacher having:
a handle portion with a plurality of openings arranged along a first direction, wherein each of the plurality of openings is sized and positioned to receive a user's finger, and
an elongate body extending from the handle portion in a second direction a substantially perpendicular to the first direction, wherein the elongate body is tapered in a distal direction along the second direction, and wherein a distal tip of the elongate body is substantially flat and beveled,
a depth gauge having an elongate body with
one or more grooves at a proximal end portion of the depth gauge, wherein the one or more grooves are configured to facilitate handheld grip of the proximal end portion,
a tapered distal end configured to facilitate insertion of the depth gauge into the subject, and
a plurality of depth markings positioned between the one or more grooves and the tapered distal end, and
a spade knife having:
a handle portion,
an elongate body extending distally from the handle portion, and
a spade-shaped blade at a distal end of the elongate body, wherein the spade-shaped blade is tapered at an angle between 6-10 degrees, and wherein the spade-shaped blade is an atraumatic blade,
wherein each of the plurality of access instruments is sized to fit at least partially into at least one of the plurality of elongated slots of the marking guide.

39. The surgical kit of claim 38, wherein the physiological parameter is a body mass index (BMI) of the subject.

40. The surgical kit of claim 38, wherein a distal edge of the spade-shaped blade of the spade knife is arched, and wherein the spade-shaped blade is sharp enough to cut and/or pierce through spinal discs and not sharp enough to cut nerves.

41. The surgical kit of claim 38, further comprising a root retractor including:
a handle portion;
an insertion portion extending from the handle portion at an obtuse angle, wherein the insertion portion has a curved cross-section configured to guide insertion of a surgical instrument along the insertion portion; and
an angled tip at a distal end of the insertion portion.

42. The surgical kit of claim 38, further comprising a root retractor bayonet including:
a handle portion;
an intermediate portion extending from the handle portion at an obtuse angle;
an insertion portion extending from the intermediate portion at a substantially right angle, wherein the insertion portion has a curved cross-section configured to guide insertion of a surgical instrument along the insertion portion; and
an angled tip at a distal end of the insertion portion.

43. The surgical kit of claim 38, further comprising a fusion guide including:
a handle portion;
an intermediate portion extending from the handle portion at an obtuse angle;
an insertion portion extending from the intermediate portion at a substantially right angle;
an angled tip at a distal end of the insertion portion; and
a non-angled tip at a distal end of the insertion portion and positioned lateral to the angled tip.

44. The surgical kit of claim 38, further comprising a pair of fusion guides including:
a first fusion guide having:
a first handle portion,
a first intermediate portion extending from the first handle portion at an obtuse angle,
a first insertion portion extending from the first intermediate portion at a substantially right angle,
a first angled tip at a distal end of the first insertion portion, and
a first non-angled tip at a distal end of the first insertion portion and positioned lateral to the first angled tip; and
a second fusion guide having:
a second handle portion,
a second intermediate portion extending from the second handle portion at an obtuse angle,
a second insertion portion extending from the second intermediate portion at a substantially right angle,
a second angled tip at a distal end of the second insertion portion, and
a second non-angled tip at a distal end of the second insertion portion and positioned lateral to the second angled tip,
wherein (i) the first angled tip and the first non-angled tip are in a first arrangement and (ii) the second angled tip and the second non-angled tip are in a second arrangement that is mirrored relative to the first arrangement.

45. The surgical kit of claim 38, further comprising a scope cannula including:
a gripping portion having a first opening sized to receive an endoscope and a second opening oriented substantially perpendicular to the first opening;
a set screw sized to fit through the second opening of the gripping portion, wherein the set screw is configured to temporarily fix a position of the endoscope relative to the gripping portion;
a tubular portion extending in distally from the gripping portion; and
an open channel portion extending distally from the tubular portion.

46. The surgical kit of claim 38, wherein the open channel has a substantially flat end.

47. The surgical kit of claim 38, wherein the open channel has a substantially arched retractor portion at a distal end thereof, wherein a concave side of the arched retractor portion faces away from a longitudinal axis of the scope cannula.

48. The surgical kit of claim 38, wherein the open channel has a substantially flat retractor portion at a distal end thereof.
